(19) United States
(12) Patent Application Publication
(10) Pub. No.: US 2024/0189201 A1
(43) Pub. Date: Jun. 13, 2024

(54) HAIR SERUM AND SUPPLEMENT

(71) Applicant: CS MEDICA A/S, Copenhagen (DK)

(72) Inventor: Lone HENRIKSEN, Greve (DK)

(21) Appl. No.: 18/286,990

(22) PCT Filed: Apr. 13, 2022
(86) PCT No.: PCT/EP2022/059861

§ 371 Date: Oct. 13, 2023

(30) Foreign Application Priority Data
Apr. 13, 2021 (DK) 2021 070171

(51) Int. Cl. A61K 8/34; A61K 31/00; A61K 31/52; A61P 17/14; A61Q 5/00

(52) U.S. Cl. CPC A61K 8/345; A61K 31/52; A61K 31/658; A61P 17/14; A61Q 5/002; A61K 2800/48

(57) ABSTRACT

The present invention relates to a hair serum and supplement for use in the treatment and/or alleviation of skin and/or hair-related symptoms and/or condition, comprising dandruff, weakening and/or breaking nails or hair, including hair-loss. Treatments may comprise application of a cannabinoid-comprising topical composition, such as a cannabidiol- and low molecular weight alcohol composition e.g. formulated as a serum, and/or consumption of a supplement comprising e.g. vitamins, zinc, selenium and amino acids, such as one or more S-comprising amino acid(s).

### FIELD OF THE INVENTION

The present invention relates to a hair serum and supplement for use in the treatment and/or alleviation of hair-, scalp-, skin-, and/or nail-related conditions in a subject. Treatments may comprise application of a cannabinoid-comprising composition, such as a cannabidiol- and low molecular weight alcohol-comprising topical composition e.g. formulated as a serum, and/or consumption of a supplement comprising e.g. vitamins, zinc, selenium and amino acids, such as one or more S-comprising amino acid(s).

### BACKGROUND OF THE INVENTION

WO2020024056 concerns composition comprising cannabidiol (CBD), ethanol and water for reducing hair loss and promoting hair growth.
CN 110664663 A (HANGZHOU AIBEIDA TECH CO LTD) relates to an anti-hair loss agent which comprises cannabidiol.
WO 2018/229072 A1 (CAPILLI MED GMBH [DE]) pertains to herbal medicin in combination with Capixyl and Rensyl for treatment of hair loss.
KARACA N. et al., J COSMO TRICHOL (2019), Vol. 5, Issue 1, “A Comparative Study between Topical 5% Minoxidil and Topical “Redensyl, Capixyl, and Procapil” Combination in Men with Androgenetic Alopecia”, concerns, inter alia, the use of Redensyl and Capixyl for treatment of hair loss.
U.S. Pat. No. 8,758,826 relates to compositions comprising CBD, ethanol, and water for the treatment of hair disorders.
WO2019049142 pertains to compositions comprising a spilanthol, a fatty acid amide isolated from Acmella oleracea and a cannabinoid, such as CBD, for treatment of Alopecia areata.

### SUMMARY OF THE INVENTION

As presented herein, surprisingly and/or unexpectedly, and from a wide range of component candidates, the inventors have found compositions comprising CBD, Redensyl and Capixyl to be effective in relation to conditions concerning or related to alpha-keratin-comprising tissue(s), such as hair-, scalp-, skin-, and/or nail-related conditions in a subject.
I. A topical, CBD- and low molecular weight alcohol-comprising composition, e.g. formulated as a serum can alleviate, lessen and/or improve such symptom(s), condition(s), or disease(s) in a subject related to one or more alpha-keratin-comprising tissues.
II. A composition for oral intake, such as supplement, can alleviate, lessen and/or improve such symptom(s), condition(s) or disease(s) in a subject related to alpha-keratin-comprising tissues. Such a composition may only require few active ingredients from a long list of possibilities, such as amino acids, vitamins, zinc and selenium (“selen”).
III. A further improvement of said symptom(s), condition(s) and/or diseases can be achieved by a combination treatment comprising application of said CBD-comprising composition with said composition for oral intake.

The present invention may comprise the following aspects, and can e.g. be summarized as follows:

In a first aspect, the present invention concerns a composition comprising:
i. Cannabidiol (CBD);
ii. Alcohol, such as a one or more low molecular weight alcohol, such as one or more C2-C4 alcohol, such a one or more of ethanol, propanol, butanol, iso-butanol, and/or any combination thereof;
iii. Water;
iv. Redensyl;
v. C3-C8 diol, such as 1,5 pentanediol;
vi. Capixyl;
vii. Menthol, and optionally
viii. Skin healing compound(s), and/or
ix. Caffeine.

Suitable compositions may be formulated as topical composition. In some embodiments, said composition may be formulated as a serum, such as a hair serum. Such a serum is considered especially suitable for scalp and/or hair-related uses and/or applications. However, it may also be applicable for treatment of other alpha-keratin-comprising tissues, such as nails and/or skin.

In some embodiments the CBD used in the provision of the topical composition is crystalline, such as “type A CBD” as disclosed herein. In some embodiments, said CBD is provided as—or capable of forming—needle-like crystals.

In a second aspect, the present invention relates to a composition for oral consumption, such as a supplement, comprising one or more vitamins, zinc, selenium, and one or more amino acids. In some embodiments, such a composition may e.g. comprise:
a. Vitamin(s); such as one or more Vitamin B(s), e.g.:
   Vitamin B5 (Panthoteic acid);
   Vitamin B6;
   Vitamin B7; and/or
   Vitamin B12; including any combination(s) thereof;
b. Zinc; such as Zinc gluconate
c. Selenium; such as L-Selenomethionine; and
d. Amino acid(s); such as one or more S-comprising amino acids, e.g.:
   L-Methionine;
   L-Cysteine; and/or
   L-Cystine; including any combination(s) thereof.

In some embodiments, such a composition can e.g. be formulated as a supplement. Apart from being suitable for treatment of scalp and/or hair-related uses and/or applications, the supplement is also suitable for nail and/or skin-related uses and/or applications.

In a third aspect, the present invention pertains to a method for providing a composition, such as topical composition according to the first aspect, e.g. a hair serum, said method comprising the acts (or steps) of:
Providing a mixture of alcohol and water, by adding alcohol directly to water;
Providing the desired ingredients in their respective amounts;
Mixing and/or dissolving the ingredients in the alcohol/water mixture;
Adjusting pH to e.g. 5-6, such as 5.5.

In some embodiments the CBD used in the provision of the topical composition is crystalline, such as “type A CBD” as disclosed herein. In some embodiments, said CBD is provided as—or capable of forming—needle-like crystals.

In a fourth aspect, the present invention concerns a composition obtained or obtainable according to a method according to the third aspect.

In a fifth aspect, the present invention relates to a topical composition for use in the treatment of a hair-, scalp-, skin-, and/or nail-related condition in a subject, wherein said treatment comprises topical application of a composition as disclosed herein, such as a CBD-comprising composition according to the first or fourth aspect to one or more of skin, hair, scalp, nail, and/or nail bed of a subject.

In a sixth aspect, the present invention pertains to a composition for oral intake, such as a supplement for use in the treatment of a hair-, skin-, scalp- and/or nail-related condition in a subject, wherein said treatment comprises intake of a supplement as disclosed herein, such as according to the second aspect.

In a seventh aspect, the present invention concerns a method of treatment a hair-, skin-, scalp- and/or nail-related condition, comprising topical application of a composition, such as a topical composition as disclosed herein, e.g. according to the first, fourth or fifth aspect, to one or more of skin, hair, scalp, nail, nail bed of a subject. In some embodiments, said treatment may comprise intake of an oral dosage form, such as a supplement. In some embodiments, said oral dosage form, e.g. supplement, is a composition as disclosed herein, such as according to the second or sixth aspect.

In an eight aspect, the present invention concerns a method of treatment a hair-, skin-, scalp- and/or nail-related condition, said method comprising intake of an oral dosage form, such as supplement as disclosed herein, e.g. according to the second or sixth aspect. Such treatment may further comprise application of topical composition, such as a cannabinoid-comprising, e.g. a CBD-comprising composition as disclosed herein, e.g. according to the first, fourth or fifth aspect, to said hair, skin, scalp and/or nail.

In a ninth aspect, the present invention pertains to one or more composition(s) and/or method(s) as disclosed herein, e.g. a CBD-comprising composition for treatment according to the fifth aspect; an oral dosage form for treatment according to the sixth aspect; and/or a method according to the seventh or eighth aspect, wherein said treatment is one or more of: hair treatment, skin treatment, and/or nail treatment.

In a tenth aspect, the present invention relates to a receptacle comprising a composition according to any one of the preceding aspects.

In an eleventh aspect, the present invention pertains to a kit comprising one or more receptacle(s) according to the tenth aspect, an instruction for use, and optionally a packaging.

In a twelfth aspect, the present invention concerns a CBD-comprising composition, such as a topical composition, wherein the CBD used in the formulation is crystalline. In some embodiments, said composition is a topical composition as disclosed herein, and/or in the third and/or fourth aspect. In some embodiments, the CBD is of type A (needle-like crystals) or capable of forming needle-like crystals, as disclosed in e.g. first aspect and/or in the Examples.

In a thirteenth aspect, the present invention pertains to a dosage regimen, comprising administering an oral supplement as disclosed herein in combination with a topical composition, in particular CBD-comprising topical composition as disclosed herein. In some embodiments, the CBD is of “type A”.

### DESCRIPTION OF THE DRAWINGS/FIGURES

FIG. 1: microscope picture of cannabinol (CBD) forming needle-like crystals. The CBD crystals were sourced from www.enecta.com.

FIG. 2: microscope picture of cannabinol (CBD) forming cluster- or bunch-like crystals. The CBD crystals were sourced from www.pharma-hemp.com.

### DETAILED DESCRIPTION OF THE INVENTION

### Definitions

In the context of the present invention, the singular form of a word may include the plural, and vice versa, unless the context clearly dictates otherwise. Thus, the references “a,” “an” and “the” are generally inclusive of the plurals of the respective terms. For example, reference to “an ingredient” or “a method” may include a plurality of such “ingredients” or “methods.”

Similarly, the words “comprise,” “comprises,” and “comprising” are to be interpreted inclusively rather than exclusively. Embodiments provided by the present disclosure may lack any element that is not specifically disclosed herein. Thus, a disclosure of an embodiment defined using the term “comprising” is also a disclosure of embodiments “consisting essentially of” and “consisting of the disclosed components”. Thus, the term “comprising” is generally to be interpreted as specifying the presence of the stated parts, steps, features, or components, but does not exclude the presence of one or more additional parts, steps, features, or components. For example, a composition comprising a chemical compound may thus comprise additional chemical compounds.

Generally, compositions as disclosed herein, in particular topical compositions and/or compositions for oral consumption may comprise one or more pharmaceutically acceptable carrier(s), excipient(s), stabilizer(s) or the like.

Where used herein, terms like “for example”, “e.g.” or “such as”, particularly when followed by a listing of terms, is merely exemplary and illustrative, and should not be deemed to be exclusive or comprehensive. Any embodiment disclosed herein may be combined with any other embodiment disclosed herein.

Unless expressed otherwise, all percentages expressed herein are by weight of the total weight of the composition. Thus, unless indicated otherwise, “%” indicates “% weight/weight (w/w)”, also called “weight %” or “% by weight”.

In the context of the present invention, the terms “about”, “around”, “approximately” or the symbol “˜” can be used interchangeably, and are meant to comprise variations and/or uncertainties generally accepted in the field, e.g. comprising analytical errors and the like. Thus “about” may also indicate measuring uncertainty commonly experienced in the art, which can be in the order of magnitude of e.g. +/−1, 2, 5, 10, or even 20 percent (%). Furthermore, “about” may be understood to refer to numbers in a range of numerals, for example the range of +/−20, +/−15, +/−10, +/−5, +/−2, +/−1, +/−0.5, +/−0.1% of the referenced number. Moreover, all numerical ranges herein should be understood to include all integers, whole or fractions, within the range.

As used herein, the term “in some embodiments” is meant to comprise “in one embodiment”, “in some embodiments”, and “in one or more embodiments”.

In the context of the present invention, the terms “subject” or “patient” can be used interchangeably, and are meant to comprise a human, animal and/or mammal. In particular, a human subject can e.g. be selected from one or more of: female, male, senior, adult, adolescent, child, or infant. An animal subject can e.g. be selected from pet, husbandry, mammal, reptile, bird, and/or animal in a zoo.

In some embodiments, the subject or patient may be diagnosed with, affected by and/or suffer from one or more symptom(s), condition(s) and/or disease(s) related to and/or manifested in an alpha-keratin-comprising tissue, such as one or more of nail, hair, and/or skin. If the subject is an animal, said alpha-keratin comprising tissue can e.g. be one or of claws, hooves, horns, and antlers.

In the context of the present invention, the term “treatment” is meant as an act aiming at alleviating, lessen, improving and/or curing any symptom(s), condition(s), or disease(s) in a subject.

Generally, “hair” is primarily composed of protein, notably alpha-keratin. Hair is a protein filament that grows from follicles found in the dermis. Hair can be seen as a defining characteristic of mammals. The human body, apart from areas of glabrous skin, is covered in follicles which produce thick terminal and fine vellus hair. Generally, the term “hair” is meant to comprise the human hair growing from the scalp, but also the fur of an animal, thus comprising down hair (also called underfur or down hair), awn hair and guard hair. In some embodiments, “hair” is the thick terminal growing on the scalp. In some embodiments, “hair” is or may comprise body hair, such as vellus, axillary, pubic hair, facial, chest abdominal, arm and/or foot hair.

Generally, a “nail” is a keratinous plate at the tip of the fingers and toes generally common to primates. Nails can be considered to be corresponding to claws in other animals. Fingernails and toenails are made of a tough protective protein called alpha-keratin which is a polymer and found in the hooves, hair, claws and horns of vertebrates. In the context of the present invention, the term “nail” is meant to comprise corresponding and/or similar tissue in an animal, such as claw, hoof, or the like.

In the context of the present invention, the term “scalp” is meant to comprise the anatomical area bordered by the human face at the front, and by the neck at the sides and back. The scalp is usually described as having five layers: (1) The skin on the head from which head hair grows. It contains numerous sebaceous glands and hair follicles. (2) The connective tissue, a dense subcutaneous layer of fat and fibrous tissue that lies beneath the skin, containing the nerves and vessels of the scalp. (3) The aponeurosis called epicranial aponeurosis (or galea aponeurotica) is the next layer. It is a tough layer of dense fibrous tissue which runs from the frontalis muscle anteriorly to the occipitalis posteriorly. (4) The loose areolar connective tissue layer provides an easy plane of separation between the upper three layers and the pericranium. (5) The pericranium is the periosteum of the skull bones and provides nutrition to the bone and the capacity for repair. In the context of the present invention, the term “scalp” is meant to comprise the skin from which thick, thermal hair grows, in particular in animals.

“Dandruff” is a skin condition that mainly affects the scalp. Symptoms include flaking and sometimes itchiness. A more severe form of the condition, which includes inflammation of the skin, is known as seborrhoeic dermatitis. The cause for dandruff is unclear, and it is believed to involve a number of genetic and environmental factors. Generally, the underlying mechanism involves the excessive growth of skin cells. To the best of the inventor's knowledge, there is no reliable cure for dandruff.

In the context of the present invention, symptom(s), condition(s), or disease(s) related to and/or manifested in alpha-keratin-comprising tissues may comprise one or more of:
i. Nails: weakening nails, breaking nails, splitting nails;
ii. Hair: weakening hair, breaking hair, reduced hair stylability, splitting hair ends, reduced hair strength, reduced hair volume, hair-loss, hair-thinning (e.g. one or more of: receding front line, loss of hair on the crown of the scalp, diffuse thinning of the hair across the entire scalp), alopecia, alopecia areata, alopecia traction, telogen effluvium, anagen effluvium, baldness, male-pattern baldness, female-pattern baldness, inherited baldness, inherited hair thinning; and/or
iii. Skin/scalp: Dandruff, seborrhoeic dermatitis, psoriasis, itchiness, burning sensation, dryness, greasiness, rash.

### Conditions Related to Hair Loss and Thinning Hair in Men:

Androgenic alopecia is considered the most common form of baldness for men and is caused by a number of factors linked to the hormone DHT where miniaturization of the hair follicles causes varying degrees of male pattern baldness. Heredity plays an important factor in the disease, as in women.

Telogen effluvium (TE)—hair loss man; if the subject is exposed to something traumatic such as malnutrition, infection, major surgeries, or other forms of extreme stress, many of the approx. 90 percent of the hairs that are in the anagen (growing) phase or the catagen (resting) phase switch over to a repulsive (telogen) phase. About 6 weeks to three months after the stressful event is usually the time when the phenomenon called Telogen Effluvium begins. It is possible to lose handfuls of hair at a time when the TE phase is at its peak. For most people who suffer from stress related hair loss (TE), hair loss is likely to continue as long as the body is affected by stress. For some men, Telogen Effluvium may be a chronic disorder and last for months or even years without any true understanding of any triggers or stressors.

Anagen effluvium—hair loss man; Anagen effluvium occurs when the hair follicle is affected from within and the hair follicle's minoic or metabolic activity is impaired. Hair loss of this type is often associated with chemotherapy. As chemotherapy targets the body's rapidly dividing cancer cells, the body's other rapidly dividing cells, such as the hair follicles in the growing (anagen) phase, will also be severely affected. Immediately after starting chemotherapy, approx. 90 percent or more of the hairs fall out while the hairs are still in the anagen phase.

### Conditions Related to Hair Loss and Thinning Hair in Women:

Androgenic alopecia—Generally, women with Androgen alopecia have diffuse thinning on all areas of the scalp. Men, on the other hand, rarely have diffuse thinning, but instead have several distinct patterns of baldness. Some women may have a combination of two pattern types. Androgenic alopecia in women is due to the action of androgens, male hormones, which are typically present in only small amounts. Androgenic alopecia can be caused by a number of factors associated with the actions of hormones, including ovarian cysts, withdrawal of high androgen index birth control pills, pregnancy and menopause. As with men, the hormone DHT may be to blame for the miniaturization of the hair follicles in women suffering from female pattern baldness. However, this is most often seen only in the 50's age and later. Heredity plays an important factor in the disease/condition.

Telogen effluvium (TE)—When the subject goes through something traumatic like childbirth, malnutrition, a serious infection, major surgeries, or extreme stress, many of the approx. 90 percent of the hairs that are in the anagen (growing) phase or catagen (resting) phase switch over to a repulsive (telogen) phase. About 6 weeks to three months after the stressful event is usually the time when the phenomenon called telogen effluvium begins. It is possible to lose handfuls of hair at a time when the TE phase is at its peak. For most people who suffer from stress related hair loss (TE), the hair loss is likely to continue as long as the body is affected by stress. For some women, Telogen effluvium may be a chronic disorder and last for months or even years without any true understanding of any triggers or stressors.

Anagen effluvium occurs when affected from within the hair follicle, thereby impairing the mitotic or metabolic activity of the hair follicle. Hair loss of this type is often associated with chemotherapy. As chemotherapy targets the body's rapidly dividing cancer cells, the body's other rapidly dividing cells, such as the hair follicles in the growing (anagen) phase, will also be severely affected. Immediately after starting chemotherapy, approx. 90 percent or more of the hairs fall out while the hairs are still in the anagen phase.

Alopecia traction is usually seen only in women and is caused by trauma inflicted on the hair follicles from tight hairstyles that pull on the hair over time. If the condition is detected and corrected early enough, the hair will be able to grow back. Traction alopecia can occur with braids, ponytails, and other tight styling hairstyles.

In a first aspect, the present invention concerns a composition comprising:
i. Cannabidiol (CBD);
ii. Alcohol, such as a one or more low molecular weight alcohol, such as one or more C2-C4 alcohol, such a one or more of ethanol, propanol, butanol, iso-butanol, and/or any combination thereof;
iii. Water; and one or more of
iv. Redensyl;
v. C3-C8 diol, such as 1,5 pentanediol;
vi. Capixyl; and/or
vii. Menthol.

Optionally, said composition may further comprise (viii) skin healing compound(s) and/or (ix) caffeine. Such a composition may thus comprise: (I) components (i)-(vi) or (i)-(vii), but neither skin healing compound(s) nor caffeine; (II) components (i)-(vi) or (i)-(vii) and skin healing compound(s) but no caffeine; (III) components (i)-(vi) or (i)-(vii) and caffeine but no skin healing compound(s); and (IV) components (i)-(vi) or (i)-(vii), skin healing compound(s) and caffeine.

Suitable concentration ranges for CBD may e.g. comprise: 0.001-2.0, 0.05-0.5, 0.075-0.2, or around 0.1% CBD by weight. In some embodiments, the concentration can also be lower than 0.001. In some embodiments, the concentration can also be greater than 2.0.

CBD is believed to provide a positive effect, such as one or more of: (i) cell replenishment/renewal; (ii) stimulation and/or increase of hair growth; and/or (iii) Dihydrotestosterone (DHT) reduction and/or blockage. DHT is believed to be involved in processes reducing the growth phase of the hair due to lowering the energy level, thus resulting in hair loss. The hair follicles gradually become smaller and are finally destroyed so that over time, baldness develops.

Generally, a CBD-comprising composition according to the first aspect comprises water and alcohol (also called the CBD-comprising composition herein), such as a one or more low molecular weight alcohol, such as one or more C2-C4 alcohol, such a one or more of ethanol, propanol, butanol, iso-butanol, and/or any combination thereof. Usually, the alcohol is miscible with water, and provides and/or contributes to solubilisation of CBD, and optionally any one or more further component. In some embodiments, the low molecular weight alcohol is or comprises a C2, C3 and/or C4 alcohol. In some embodiments, the low molecular weight alcohol is or comprises a C2 or C3 alcohol. In some embodiment, the low molecular weight alcohol is or comprises ethanol.

Suitable concentration ranges for alcohol may e.g. comprise: 10-60, 30-55, 40-50, or around 30% alcohol; such as low molecular weight alcohol, e.g. ethanol by weight. In some embodiments, the concentration can also be lower than 10%. In some embodiments, the concentration can also be greater than 60%.

In the context of the present invention, it is believed that the low molecular weight alcohol, such as ethanol works as a penetration enhancer, thus contributing, mediating and/or facilitating provision of active substances into the skin and/or scalp. In some embodiments, the low molecular ethanol may act as a penetration enhancer, sorption promotor and/or accelerant.

The CBD-comprising composition comprises water, usually provided up to 100% by weight. Water may act as a solvent, dilutant, carrier, and/or vehicle for one or more active ingredients. Generally, water is also believed less irritant and more “skin-” and or “hair-friendly” than alcohol, thus reducing mitigating an undesirable effect of e.g. alcohol and/or other components.

Suitable concentration ranges for water may e.g. comprise: 30-70, 35-60, 40-55, or around 45% water by weight. Water can also be provided “up to 100%” in the final composition. In some embodiments, the concentration can also be lower than 30%. In some embodiments, the concentration can also be greater than 70%.

In some embodiments, a CBD-comprising composition comprises Redensyl=component (iv). In some embodiments, a CBD- and low molecular weight alcohol-comprising composition comprises component (iv), and one or more of components (v), (vi), and/or (vii).

Suitable concentration ranges for Redensyl may e.g. comprise 0.5-6.0, 1.0-5.0, 2.0-4.0, or around 3% Redensyl by weight. In some embodiments, the concentration can also be lower than 0.5%. In some embodiments, the concentration can also be greater than 6.0%.

Redensyl is believed to provide hair growth-stimulating effect. Without wanting to be bound by any theory, it is believed that the combination of CBD and Redensyl provides a positive, synergistic effect. Redensyl is reported to comprise, consist, or to consist essentially of: Dihydroquercetin-glucoside (DHQG: 0.005%), Epigallocatechin gallate-glucoside (EGCG2: 0.0009%), Glycine (0.005%), Zinc chloride (0.002%), Meta-bisulfite (0.015%), Glycerin: 50%, and Water QSP 100%. More info concerning Redensyl® can e.g. be found here:
http://www.google.com/url?sa=t&rct=j&q=&esrc=s&source=web&cd=&cad=rja&uact=8&ved=2ahUKEwjUzM3s2JbvAhVItIsKHXmuAyoQFjABegQIARAD&url=http %3A %2F % 2Fwww.nardev.com %2FUploadSection % 2FProdCat-276-1446515551.pdf&usg=AOvVaw26ucjjgXwnMona4A9m-t_I, or www.gomo-energy.com/media/pdf/ae/c6/32/redensyl_studie.pdf

In some embodiments, a CBD-comprising composition comprises a diol, such as a C3-C8 diol, e.g. 1,5 pentanediol=component (v). In some embodiments, a CBD- and low molecular weight alcohol-comprising composition comprises component (v), and one or more of components (iv), (vi), and/or (vii).

Suitable concentration ranges for C3-C8 diol may e.g. comprise: 0.5-5.0, 1.0-4.0, 1.5-2.5, or around 2% C3-C8 diol, e.g. 1,5 pentanediol by weight. In some embodiments, the concentration can also be lower than 0.5%. In some embodiments, the concentration can also be greater than 5.0%.

It is believed that the diol, such as C3-C8 diol, e.g. 1,5 pentanediol may act as a solvent, thereby providing a cleaning/purifying effect on obstructed hair sacks. Furthermore, the diol may also act as a penetration enhancer, sorption promotor and/or accelerant.

In some embodiments, a CBD-comprising composition comprises Capixyl=component (vi). In some embodiments, a CBD- and low molecular weight alcohol-comprising composition comprises component (vi), and one or more of components (iv), (v), and/or (vii).

Suitable concentration ranges for Capixyl may e.g. comprise: 0.1-2.0, or 0.2-1.0, 0.4-0.8, around 0.57% Capixyl by weight. In some embodiments, the concentration can also be lower than 0.1%. In some embodiments, the concentration can also be greater than 2.0%, such as 2.5% or more, or 3.0 or more.

Capixyl is believed to provide an increased and/or stimulated hair growth, and/or as a DHT blocker. Without wanting to be bound by any theory, it is believed that the combination of CBD and Capixyl provides a positive, synergistic effect. Capixyl™ is reported to comprise, consist, or consist essentially of (INCI) Butylene Glycol, Aqua, Dextran, Acetyl Tetrapeptide-3, Trifolium Pratense (Clover) Flower Extract. More details can e.g. be found here: https://www.lucasmeyercosmetics.com/sites/lucasmeyer-corp-v2/files/products/pdf/6-tech-file-capixyl.pdf, or www.foligain.nl/media/downloads/Capixyl.pdf.

In some embodiments, a CBD-comprising composition comprises menthol=component (vii). In some embodiments, a CBD- and low molecular weight alcohol-comprising composition comprises component (vii), and one or more of components (iv), (v), and/or (vi).

Suitable concentration ranges for menthol may e.g. comprise: 0.1-2.0, 0.2-1.0, 0.4-0.8, or around 0.6% menthol by weight. In some embodiments, the concentration can also be lower than 0.1%. In some embodiments, the concentration can also be greater than 2.0%.

It is believed that menthol stimulates blood flow and/or circulation in the skin and/or scalp, in particular in the smaller blood vessels in the skin/scalp.

Optionally, one or more further components may be present, such as e.g.
viii. Skin healing compound(s), and/or
ix. Caffeine.

Thus, in some embodiments, a CBD-comprising composition as disclose above may further comprise one or more skin healing compound(s)=component (viii).

Suitable concentration ranges for skin healing compound(s) may e.g. comprise: 0.01-3.0, 0.1-2.0, 0.5-1.5, or around 1.0% skin healing compound(s) by weight. In some embodiments, the concentration can also be lower than 0.1%. In some embodiments, the concentration can also be greater than 3.0%.

It is believed that the effect of a skin healing compound is one or more of: increased skin healing, improved repair of skin lesions, and/or wound healing.

In some embodiments, said skin healing compound is, or comprises Hyaluronic acid/Hyaluronate. In some embodiments, said CBD-comprising composition comprises 0.01-2.0, 0.1-1.5, 0.85-1.15, or around 0.95% Skin healing compound(s), such as Hyaluronic acid/Hyaluronate.

It is believed that Hyaluronic acid/salt, such as sodium hyaluronate contributes to keeping the hair shaft moist, and/or making it easier to comb or brush the hair, which in turn decreases breakage. Furthermore, Hyaluronic acid/salt, such Na hyaluronate may provide one or more of: stimulation of hair growth; exfoliating dry scalp; promoting body, resilience, and softness of the hair, and/or supporting and strengthening weaker hair strands.

Furthermore, in some embodiments, a CBD-comprising composition as disclosed above may comprise caffeine=component (vii), and one or more of components (iv), (v), and/or (vi).

Suitable concentration ranges for caffeine may e.g. comprise: 0.01-2.0%, 0.02-0.5, 0.04-0.07, or around 0.05% caffeine by weight. In some embodiments, the concentration can also be lower than 0.01%. In some embodiments, the concentration can also be greater than 2.0%.

The effect of caffeine is believed to comprise its biological activity and ability to penetrate the skin barrier. Caffeine is also believed to increase the microcirculation of blood in the skin/scalp. Caffeine is also believed to stimulate hair growth, such as through inhibition of 5-alpha reductase activity, and enzyme capable of converting Testosterone to DHT.

Suitably, compositions can be formulated as topical composition. In some embodiments, said composition may be formulated as a serum, such as a hair serum. Such a serum is considered especially suitable for scalp and/or hair-related uses and/or applications. The term “serum” may not be defined clearly in the art, but it seems generally accepted that a skin- or hair serum are skin and/or hair care products that are designed to deliver high concentrations of specific active ingredients to the skin, and in particular to the scalp in the context of a hair serum. Often, serums appear clear, they can be gel-based or liquid, and they tend to be less thick than a moisturizer. It is believed that relevant active components/molecules are able to penetrate the skin. Generally, active ingredient(s) in serums tend to be more concentrated than in conventional non-serum formulations. Thus, great care has to be taken to avoid undesired reactions, such as irritation and/or dryness upon treatment with a serum, in particular when the subject possesses a sensitive skin. It is believed that the CBD-comprising compositions according to the first aspect are designed and/or formulated to avoid such negative reactions.

In some embodiments, a CBD-comprising composition as disclosed herein, such as a hair serum may also provide a positive effect on a nail-related condition. This can e.g. be achieved simultaneously when applying the hair serum to the scalp and massaging the composition into scalp and/hair with the fingers.

Thus, in some embodiments, a CBD-comprising composition as disclose above may further comprise one or more further compound(s), such as one or more components selected form components (x) to (xviii), wherein
x. =Propylene glycol;
xi. =Glycerine;
xii. =Skin conditioning agent(s), emollient and/or binder, such as Isopropyl palmitate;
xiii. =Triethanolamine;
xiv. =Arginine;
xv. =Panthenol;
xvi. =Vitamin(s), such as Vitamin B, e.g. Vitamin B7 (Biotin);
xvii. =pH adjusting agent (s) such as citric acid monohydrate;
xviii. =Thickening agent(s), such as Xanthan gum.

In some embodiments, a CBD-comprising composition as disclose above may further comprise propylene glycol=component (x); and optionally one or more of components (xi-xviii).

Suitable concentration ranges for propylene glycol may e.g. comprise: 1.0-20, 2-10, 4-6, or around 5.0% propylene glycol by weight. In some embodiments, the concentration can also be lower than 1.0%. In some embodiments, the concentration can also be greater than 20%.

It is believed that propylene glycol and/or closely related compounds provide a humectant (hydrating) effect and/or may act as penetration enhancer.

In some embodiments, a CBD-comprising composition as disclose above may further comprise glycerine=component (xi); and optionally one or more of components (x, xii-xviii).

Suitable concentration ranges for glycerine may e.g. comprise: 1.0-20, 2-15, 5-8, or around 6.5% glycerine by weight. In some embodiments, the concentration can also be lower than 1.0%. In some embodiments, the concentration can also be greater than 20%.

Glycerine is believed to provide a humectant effect, and/or may acts as a type of moisturizing agent that “pulls” water into the outer layer of the skin/scalp.

In some embodiments, a CBD-comprising composition as disclosed above may further comprise one or more skin conditioning agent(s), emollient(s) and/or binder(s), including any combination thereof, such as isopropyl palmitate=component (xii); and optionally one or more of components (x, xi, xiii-xviii).

Suitable concentration ranges for skin conditioning agent(s), emollient(s) and/or binder(s), including any combination thereof, such as isopropyl palmitate may e.g. comprise: 0.05-2.0, 0.1-1.0, 0.7-1.2, or 0.9% skin conditioning agent(s), emollient(s) and/or binder(s), including any combination thereof, such as isopropyl palmitate by weight. In some embodiments, the concentration can also be lower than 0.05%. In some embodiments, the concentration can also be greater than 2.0%.

Isopropyl palmitate is believed to act as a moisturizer by helping to bind moisture to the skin. It is also an emollient, which helps products spread smoothly, and it can be used as a fragrance ingredient. Generally, conditioning agent(s) is/are believed to provide a positive effect, such as a skin caring effect. Isopropyl palmitate and/or related compound is/are believed to provide a skin conditioning effect, such as acting as moisturizer and/or emollient. Isopropyl Palmitate may also function as a binder thus providing a positive effect when formulating the composition, and/or with respect to its storage stability.

In some embodiments, a CBD-comprising composition as disclose above may further comprise triethanolamine=component (xiii); and optionally one or more of components (x-xii, xiv-xviii).

Suitable concentration ranges for triethanolamine may e.g. comprise: 0.1-2.0, 0.15-1.0, 0.2-0.4, or around 0.28% triethanolamine by weight. In some embodiments, the concentration can also be lower than 0.1%. In some embodiments, the concentration can also be greater than 2.0%.

Triethanolamine is believed to function as surfactant; it may also function as pH adjustment agent, hair fixatives, and may possess a preservative effect. Thus Triethanolamine (or related compounds) may comprise one or more positive effect in a formulation as disclosed herein.

In some embodiments, a CBD-comprising composition as disclose above may further comprise Arginine=component (xiv); and optionally one or more of components (x-xiii, xv-xviii).

Suitable concentration ranges for arginine may e.g. comprise: 0.0001-1.0, 0.0005-0.1, 0.001-0.003, or around 0.002% arginine by weight. In some embodiments, the concentration can also be lower than 0.0001%. In some embodiments, the concentration can also be greater than 1.0%.

Arginine is believed to relax blood vessels, thereby improving the flow of the blood to the skin and/or scalp and the base of hair follicles. This is also believed to provide a hair growth-stimulating and/or hair regenerating effect. It is believed that the presence of arginine in a topical composition may prevent poor development of hair follicles, and/or promote healthier hair.

In some embodiments, a CBD-comprising composition as disclose above may further comprise panthenol=component (xv); and optionally one or more of components (x-xiv, xvi-xviii).

Suitable concentration ranges for panthenol may e.g. comprise: 0.5-5.0, 1.0-4.0, 2.5-3.0, or around 2.7% panthenol by weight. In some embodiments, the concentration can also be lower than 0.5%. In some embodiments, the concentration can also be greater than 5.0%.

Panthenol is believed to strengthen hair, retain moisture, and/or improve the texture of damaged hair.

In some embodiments, a CBD-comprising composition as disclose above may further comprise Vitamin(s), such as Vitamin B, e.g. Vitamin B7 (Biotin)=component (xvi); and optionally one or more of components (x-xv, xvii, xviii).

Suitable concentration ranges for vitamin(s) may e.g. comprise: 0.1-2.0, 0.2-1.5, 0.8-1.2, or around 1.0% Vitamin by weight. In some embodiments, the concentration can also be lower than 0.1%. In some embodiments, the concentration can also be greater than 2.0%.

Vitamins, in particular vitamin B, such as Biotin are believed to be related to health of hair, skin and/or nails. In particular, a lack of such vitamin(s) may lead to hair loss and or skin conditions, such as scaly red rash.

In some embodiments, a CBD-comprising composition as disclosed above may further comprise pH adjusting agent (s) such as citric acid monohydrate=component (xvii); and optionally one or more of components (x-xvi, xviii).

Suitable concentration ranges for pH adjusting agent(s) may e.g. comprise: 0.1-2.0, 0.2-1.0, 0.25-0.5, or around 0.4% pH adjusting agent(s), such as citric acid monohydrate. In some embodiments, the concentration can also be lower than 0.1%. In some embodiments, the concentration can also be greater than 2.0%.

Apart from providing a pH-adjusting/stabilizing effect, citric acid (or salts thereof, such as ammonium, potassium and/or sodium salts) may also provide one or more further effects, such as an anti-oxidant, anti-microbial, thickening, and/or chelating effect, including any combinations thereof. In some embodiments, the citric acid/citrate provides an antioxidant effect. In some embodiments, the citric acid/citrate provides an anti-microbial effect. In some embodiments, the citric acid/citrate may also have a thickening effect on the serum. In some embodiments, the citric acid/citrate may also have a chelating effect, such as by complexing metal ions.

In some embodiments, a CBD-comprising composition as disclosed above may further comprise thickening agent(s), such as xanthan gum=component (xviii); and optionally one or more of components (x-xvii).

Suitable concentration ranges for thickening agent(s), such as Xanthan Gum may e.g. comprise: 0.1-2.0, 0.15-1.0, 0.15-0.4, or around 0.25% thickening agent(s) by weight. In some embodiments, the concentration can also be lower than 0.1%. In some embodiments, the concentration can also be greater than 2.0%.

Generally, thickening agent may provide a viscosity increasing effect. Such an agent, e.g. Xanthan Gum may furthermore function as a one or more of: binder, emulsion stabilizer, skin-conditioning agent, surfactant, emulsifying agent, including any combination thereof.

In summary, in some embodiments of a CBD-comprising composition according to the first aspect, one or more components can be provided in a concentration (% by weight) of:
i. 0.001-2.0, 0.05-0.5, 0.075-0.2, or around 0.1% CBD;
ii. 10-60, 30-55, 40-50, or around 30% alcohol; such as EtOH
iii. 30-70, 35-60, 40-55, or around 45% Water; and/or water up to 100%;
iv. 0.5-6.0, 1.0-5.0, 2.0-4.0, or around 3% Redensyl;
v. 0.5-5.0, 1.0-4.0, 1.5-2.5, or around 2% C3-C8 diol;
vi. 0.1-2.0, or 0.2-1.0, 0.4-0.8, around 0.57% Capixyl;
vii. 0.1-2.0, 0.2-1.0, 0.4-0.8, or around 0.6% Menthol;
viii. 0.01-2.0, 0.1-1.5, 0.85-1.15, or around 0.95% % Skin healing compound(s);
ix. 0.01-2.0%, 0.02-0.5, 0.04-0.07, or around 0.05% Caffeine;
x. 1.0-20, 2-10, 4-6, or around 5.0% Propylene glycol;
xi. 1.0-20, 2-15, 5-8, or around 6.5% Glycerine;
xii. 0.05-2.0, 0.1-1.0, 0.7-1.2, or 0.9% Isopropyl palmitate;
xiii. 0.1-2.0, 0.15-1.0, 0.2-0.4, or around 0.28% Triethanolamine;
xiv. 0.0001-1.0, 0.0005-0.1, 0.001-0.003, or around 0.002% Arginine;
xv. 0.5-5.0, 1.0-4.0, 2.5-3.0, or around 2.7% Panthenol;
xvi. 0.1-2.0, 0.2-1.5, 0.8-1.2, or around 1.0% Vitamin;
xvii. 0.1-2.0, 0.2-1.0, 0.25-0.5, or around 0.4% pH adjusting agent(s);
xviii. 0.1-2.0, 0.15-1.0, 0.15-0.4, or around 0.25% thickening agent(s),
   to provide a CBD-comprising topical composition according to the first aspect. As mentioned herein, such compositions are often formulated with a near neutral, and/or slightly acidic pH, such as pH 5-7.5 or 5.5-6.5.

In some embodiments, at least 3, 4, 5, 6, 7, or 8 of components (i)-(ix) are provided in concentration as disclosed above.

In some embodiments, at least 10, 11, 12, 13, 14, 15, 16, or 17 of components (i)-(xviii) are provided in concentration as disclosed above.

In some embodiments, a CBD-comprising composition is formulated such that a defined pH is provided. Generally, a neutral, near neutral, and/or slightly acidic pH, such as mimicking the pH of the skin or scalp is considered advantageous. In some embodiments, a CBD-comprising composition can be formulated with a pH of 5-7, 5-6, 5.5-6.5, or around 6. In some embodiment, the pH is around 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 0.6.9, or 7.0. In some embodiments, the pH can be around 5.2-5.8, 5.6-5.7 or around 5.5. In some embodiments, the pH is lower than 5.0. In some embodiments, the pH is greater than 7.0.

Provision of a defined pH can be achieved using methods known in the field, comprising addition of one or more acid(s), base(s), salt(s) of said acid(s) and/or base(s), and/or buffering agent(s), including any combination thereof. In some embodiments, citric acid, in particular citric acid monohydrate is used in this context. In some embodiments, other pharmaceutically acceptable acid(s) or base(s) including their salts can be used. In some embodiments, triethanolamine and/or citrate/citric acid are used in the provision and/or maintenance of the desired pH.

Generally, the presence of oil(s) and/or fat(s) is not desired in CBD-comprising compositions according to the invention, in particular in the context of a topical composition to be applied to the hair and/or scalp of a subject. Without wanting to be bound by any theory, it is believed that such fat(s) and/or oil(s) may contribute negatively with respect to e.g. customer/patient satisfaction, such as by causing a greasy scalp and/or hair. Thus, in some embodiments, a CBD-comprising composition as disclosed herein comprises less than 1.0 or 0.1% oil(s) and/or fat(s).

In particular, when absence of oil(s) and/or fat(s) is desired, common sources of CBD, such as CBD-comprising oils are not desirable. Thus, in some embodiment, CBD is provided in essentially pure form, such as in crystalline or powder form and/or with a purity of 95%, 98%, 99%, or more than 99% or more. Without wanting to be bound by any theory, it is believed that the use of CBD in crystalline may further contribute in a positive fashion, such as that less CBD is required to provide a similar effect compared to a crude CBD preparation. This is surprising, as according to general belief, further cannabinoids present in such crude CBD preparations are believed to provide a synergistic effect.

Consequently, in some embodiments, the CBD-comprising composition does not comprise one or more further cannabinoid(s), such as one or more hallucinogenic and/or non-hallucinogenic cannabinoid. In some embodiments, no further cannabinoid(s) is/are present in amount above 10, 5, 2, 1, 0.5, or even 0.1% by weight in relation to CBD. In some embodiments, said composition does not comprise one or more further cannabinoid(s), such as one or more hallucinogenic and/or non-hallucinogenic cannabinoid, and/or wherein said further cannabinoid(s) is/are present in amount below 10, 5, 2, 1, 0.5 or 0.1% by weight in relation to CBD.

However, in some embodiments a further cannabinoid, or even several further cannabinoids may be present.

In some embodiments, a CBD-comprising composition according to the first aspect may comprise a further cannabinoid, such as a one or more cannabinoid(s) selected from: THC (tetrahydrocannabinol), THCA (tetrahydrocannabinolic acid), CBDA (cannabidiolic acid), CBN (cannabinol), CBG (cannabigerol), CBC (cannabichromene), CBL (cannabicyclol), CBV (cannabivarin), THCC (tetrahydrocannabiorcol), THCV (tetrahydrocannabivarin), THCP (tetrahydrocannabiphorol), CBDV (cannabidivarin), CBCV (cannabichromevarin), CBGV (cannabigerovarin), CBGM (cannabigerol monomethyl ether), CBE (cannabielsoin), CBT (cannabicitran), including one or more cannabinoids of the following types: CBG-type, CBC-type, “CBD-type other than CBD”, THC-type, CBN-type, CBE-type, iso-THC-type, CBL-type, CBT-type, including any combination(s) thereof. Such further cannabinoid may comprise hallucinogenic and/or non-hallucinogenic cannabinoids. Generally, non-hallucinogenic cannabinoids are preferred in order to avoid undesired side-effects upon use or treatment with composition(s) comprising such compounds, in particular when they are present in physiologically active amounts.

It is believed that the presence of one or more further cannabinoid(s) may provide a synergistic effect. In some embodiments, said further cannabinoid may be selected from: CBDV, CBDA, CBG, CBN, THC, and CBC.

In some embodiment, said further cannabinoid can be provided in a ratio CBD:further cannabinoid of >100:1, 100:1-50:1, 50:1-25:1. 25:1.10:1, 10:1, 10:1-5:1, 5:1-2:1, 2:1-1:1, 1:1-1:2, 1:2-1:5, or <1:5. In some embodiments, said ratio CBD:further cannabinoid is around 10:1, 9:1, 8:1, 7:1, 6:1, 5:1, 4:1, 3:1, 2:1, 1.5:1, 1.25:1, 1:1, 1:1.25, 1:1.5, 1:2, 1:3, 1:4, or 1:5.

In some embodiments, the CBD is at least 98, 99, 99.5, or 99.9% pure. In some embodiments, the CBD comprises less than <1.5% any other cannabinoid. In some embodiments, the CBD comprises less than <1.5% any other cannabinoid and less than 1.0% or 0.1% THC. In some embodiments, the CBD comprises less than 0.1% by weight THC and/or any other hallucinogenic cannabinoid.

Concerning suitable topical formulations of a CBD-comprising composition according to the first aspect, in some embodiment said composition can be formulated as a hair serum. Without wanting to be bound by any theory, it is believed that a composition, such as a serum according to the present invention may provide activation of important portion(s) and/or cells in the hair follicle/root, such as stem cells. As such, the serum is formulated to allow penetration of ingredients into/towards the hair follicle and/or hair root.

Further embodiments CBD-comprising hair serums, usually formulated with a pH of 5-7.5, such as 5.5-6.5 may comprise (concentrations and/or ranges are given in % by weight):
Embodiment A:
   i. 0.001-2.0% CBD;
   ii. 10-60% alcohol; such as EtOH and
   iii. 30-70% water.
Embodiment B:
   i. 0.001-2.0% CBD;
   ii. 10-60% alcohol; such as EtOH
   iii. 30-70% water; and optionally one or more of:
   iv. 0.5-6.0% Redensyl;
   v. 0.5-5.0% C3-C8 diol;
   vi. 0.1-2.0% Capixyl; and
   vii. 0.1-2.0% Menthol.
Embodiment C:
   i. 0.001-2.0% CBD;
   ii. 10-60% alcohol; such as EtOH
   iii. 30-70% water; and optionally one or more of:
   iv. 0.5-6.0% Redensyl;
   v. 0.5-5.0% C3-C8 diol;
   vi. 0.1-2.0% Capixyl;
   vii. 0.1-2.0% Menthol;
   viii. 0.01-2.0% Skin healing compound(s); such as Hyaluronic acid/Hyaluronate; and/or
   ix. 0.01-2.0% Caffeine.
Embodiment D:
   i. 0.001-2.0% CBD;
   ii. 10-60% alcohol; such as EtOH
   iii. 30-70% water;
   iv. 0.5-6.0% Redensyl;
   v. 0.5-5.0% C3-C8 diol;
   vi. 0.1-2.0% Capixyl;
   vii. 0.1-2.0% Menthol;
   viii. 0.1-2.0% Skin healing compound(s); such as Hyaluronic acid/Hyaluronate; and/or
   ix. 0.01-2.0% Caffeine; wherein either (iv), (vi) or (iv)+(vi) are optional.
Embodiment E:
   i. 0.001-2.0% CBD;
   ii. 10-60% alcohol; such as EtOH
   iii. 30-70% water;
   iv. 0.5-6.0% Redensyl;
   v. 0.5-5.0% C3-C8 diol;
   vi. 0.1-2.0% Capixyl;
   vii. 0.1-2.0% Menthol; and optionally one or more of:
   viii. 0.1-2.0% Skin healing compound(s), such as Hyaluronic acid/Hyaluronate;
   ix. 0.01-2.0% Caffeine;
   x. 1.0-20% Propylene glycol;
   xi. 1.0-20% Glycerine;
   xii. 0.05-2.0% Skin conditioning agent(s), emollient and/or binder, such as Isopropyl palmitate;
   xiii. 0.1-2.0% Triethanolamine;
   xiv. 0.0001-1.0% Arginine;
   xv. 0.5-5.0% Panthenol;
   xvi. 0.1-2.0% Vitamin;
   xvii. 0.1-2.0% pH adjusting agent(s);
   xviii. 0.1-2.0% thickening agent(s).
Embodiment F:
   i. 0.001-2.0% CBD;
   ii. 10-60% alcohol; such as EtOH
   iii. 30-70% water;
   iv. 0.5-6.0% Redensyl;
   v. 0.5-5.0% C3-C8 diol;
   vi. 0.1-2.0% Capixyl;
   vii. 0.1-2.0% Menthol;
   viii. 0.1-2.0% Skin healing compound(s), such as Hyaluronic acid/Hyaluronate;
   ix. 0.01-2.0% Caffeine;
   x. 1.0-20% Propylene glycol;
   xi. 1.0-20% Glycerine;
   xii. 0.05-2.0% Skin conditioning agent(s), emollient and/or binder, such as Isopropyl palmitate;
   xiii. 0.1-2.0% Triethanolamine;
   xiv. 0.0001-1.0% Arginine;
   xv. 0.5-5.0% Panthenol;
   xvi. 0.1-2.0% Vitamin;
   xvii. 0.1-2.0% pH adjusting agent(s);
   xviii. 0.1-2.0% thickening agent(s).

In some embodiments, the skin healing compound (viii) is Hyaluronic acid/Hyaluronate.

In some embodiments, the skin conditioning, emollient and/or binding agent (xii) is Isopropyl palmitate.

In some embodiments, components viii and xii are Hyaluronic acid/Hyaluronate and Isopropyl palmitate, respectively.

Some of the components may be optional, as disclosed herein. In some embodiments one or more ingredient(s) may be provided in a higher or lower concentration.

Further suitable concentrations and/or concentration ranges may be disclosed herein.

Concerning the CBD used in the preparation or formulation of a CBD-comprising topical formulation, in some embodiments, the CBD used in the provision of the topical composition is crystalline.

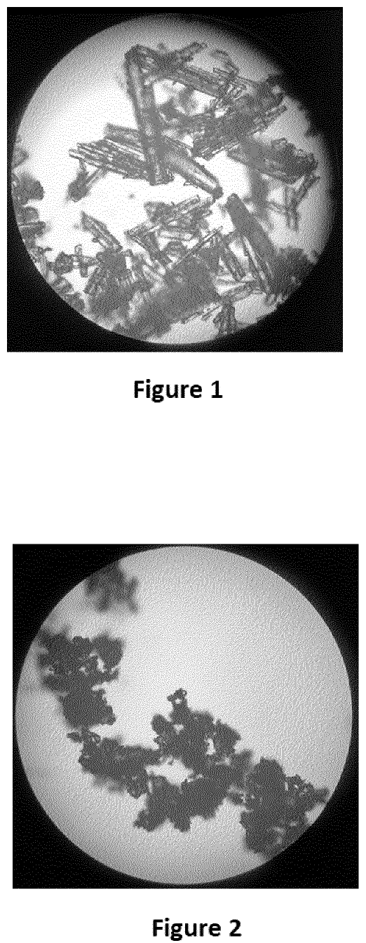

In some embodiments, the CBD used for providing a topical composition as disclosed above is characterized by a certain features, such as the crystal structure and/or conformation. It has been observed by the inventors, see e.g. Example 12, that CBD with a needle-like crystal structure (=crystal structure A; see FIG. 1), surprisingly and unexpectedly, appears significantly more potent than CBD with a different crystal structure, a non-needle like structure, also termed “bunch-like or “cluster-like” herein (=crystal structure B; see FIG. 2).

In some embodiments, the CBD possesses, when crystalline, or is capable of forming a needle-like crystal structure. In some embodiments, CBD of crystal structure A (or capable of forming needle-like crystals) is at least 1.5, 2, 3, 4, 5, 7.5, 10, 15 or 20 times more potent on a weight/weight basis than CBD of crystal structure B (or capable of forming cluster/bunch-like crystals).

CBD of crystal structure A, or CBD capable of forming needle-like crystals, is also called “type A CBD” herein, while CBD of crystal structure B, or CBD capable of forming “bunch-like or “cluster-like” crystals is called “type B CBD”. In some embodiments, the CBD is “type A CBD”. Often, “type A CBD” is preferred in contrast to “type B CBD”.

It can be speculated, if the CBD needs to be in an active form, such one or more specific conformation(s) in order to be active upon administration to a subject, such as in a topical formulation. Lack of activity or potency can also be caused by a lower uptake rate and/or difficulties in passing through the skin.

Without wanting to be bound by any theory, it is believed that the difference in crystal structure may be caused by a different molecular structure, such as a different conformation. This could e.g. be due to a failure of the subject's body to recognize the “wrong” CBD conformation or the like. It is conceivable that the differences in CBD crystal structure are caused by a different extraction process. In particular, the CBD disclosed in FIG. 1 was provided by an extraction process, comprising extraction with isopropanol, distillation and crystallization with heptane (see e.g. Example 12), while the CBD disclosed in FIG. 2 was provided by critical CO₂ extraction.

Generally, crystalline CBD can be provided by methods and techniques known in the art, such as by methods disclosed in U.S. Ser. No. 10/413,845 and/or U.S. Ser. No. 10/414,709.

In short, crystalline CBD can be provided from hemp or cannabis (Cannabis sativa) by a method consisting essentially of:
Extracting hemp or cannabis with e.g. isopropanol to produce an extract rich in cannabinoids, THC, CBD and terpenes
Evaporating the solvent portion of the extract to generate a substantially solvent-free extract
Distilling the substantially solvent-free extract to isolate the CBD, and
Crystallizing the distilled, isolated CBD to produce a crystallized, isolated CBD and one or more recrystallization(s) if needed by the use of a suitable organic solvent, such as an alkane, e.g. heptane, commonly followed by
Solvent removal by e.g. vacuum drying. to remove volatile remnants.

Thus, in some embodiments, the CBD crystals used in the formulation of the topical composition are needle-like crystals, such as crystals shown in FIG. 1. Likewise, in some embodiments, the CBD crystals used in the formulation of the topical composition are not cluster- or bunch-shaped, such as crystals similar to crystals shown in FIG. 2.

In some embodiments, the CBD crystals used in the formulation of the topical composition are not provided by an extraction method comprising critical CO₂ extraction.

In some embodiments, the CBD crystals used in the formulation of the topical composition are provided by a method comprising extraction with a C₃-C₄ alcohol, such as isopropanol, and one or more crystallisations steps with a C₆-C₈ alkane, such as heptane. In some embodiments, the C₃-C₄ alcohol is isopropanol. In some embodiments, the C₆-C₈ alkane is heptane. In some embodiments, the C₃-C₄ alcohol is isopropanol, and the C₆-C₈ alkane is heptane. This combination is believed to provide CBD crystals of satisfactory quality, such as absence or reduction in inhibitors and/or the desired conformation of the CBD.

In some embodiments, a suitable CBD product can be obtained when the CBD crystals are provided by a method comprising critical CO₂ extraction and one or more crystallisations steps with a C₆-C₈ alkane, such as heptane.

As seen in Table 1, it can be seen that the Cannabinoid profile of type A and type B CBD can be rather similar.

TABLE 1
Analysis of CBD of crystal structure A versus crystal structure B

| Cannabinoid profile | Type A | Type B |
|---|---|---|
| CBD | 99.33% | 98.60% |
| CBDV | 0.39% | 0.19% |
| CBDA | 0.01% | n.d. |
| CBG | n.d. | n.d. |
| CBN | 0.04% | n.d. |
| THC | n.d. | n.d. |

n.d. not detected;
type A CBD was sourced from Enecta, type B CBD was sourced from Pharma Hemp

It is, however, also conceivable that the differences in crystal structure, can relate to and be caused by different extraction processes. Different crystal structures can also be indicative of different concentrations of “CBD inhibitors”, and/or different concentrations of “CBD enhancers”. In some embodiments, terpenes, such as naturally occurring terpenes, in particular terpenes found in plants, such as in Cannabis sativa, act as CBD inhibitors, which is not desirable.

Thus, in some embodiments, CBD of crystal structure B alias “type B CBD” can be converted to CBD of crystal structure A alias “type A CBD” (and/or CBD capable of forming crystal structure A) by an organic extraction step and/or recrystallisation step. In such embodiments, it is conceivable that the change in crystal structure is related to the presence of inhibitors that are reduced significantly in the additional extraction and/or crystallization step(s). Alternatively, the organic extraction step may provide a change in conformation of the CBD, rendering it more active again. In some embodiments, recrystallization with heptane can change the B-type CBD into A-type CBD.

In some embodiments, CBD of crystal structure B has been provided by critical CO₂ extraction, such as CBD crystals provided by www.pharma-hemp.com and/or following a similar extraction protocol as said manufacturer.

In some embodiments, presence of terpenes and/or terpenoids, in particular Cannabis sativa terpenes or in a CBD-comprising topical composition as disclosed herein, provides one or more undesirable effect(s), such as one or more of: reduced efficiency or potency, inability or reduced ability to recognize the CBD, need for a higher CBD formulation for obtaining similar effect, increase in non-CBD cannabinoids in the formulation. In some embodiments, said composition comprises 0.0001% or less, 0.001% or less, 0.01% or less, or 0.1% or less terpenes, in particular Cannabis sativa terpenes, by weight.

In some embodiments, the crystalline CBD does not comprise significant amounts of terpenes, such as less than 0.1, less than 0.05, less than 0.02, less than 0.01, less than 0.005, less than 0.002, less than 0.001% terpenes by weight.

It is also conceivable that other plant components, such as terpenoids can act as inhibitors. In some embodiments the presence of terpenoids, such as Cannabis sativa terpenoids can be undesirable. In some embodiments, the crystalline CBD does not comprise significant amounts of terpenoids, such as less than 0.1, less than 0.05, less than 0.02, less than 0.01, less than 0.005, less than 0.002, less than 0.001% terpenoids by weight.

In some embodiments, the use of CBD having or capable of providing crystals of crystal structure A, such as shown in FIG. 1 in a CBD-comprising topical composition as disclosed herein, provides a positive effect, such as one or more of: increased efficiency, possibility to reduce total amount of CBD in the formulation, the subject needs less topical formulation to achieve the same effect, improved recognition and/or CBD uptake by the subject's body, reduction in non-CBD cannabinoids in the formulation and/or other impurities.

Generally, CBD-comprising compositions can be formulated for daily use, such as once or twice per day, preferably once per day with respect to ease of use.

Generally, compositions according to the first aspect can be provided using methods and procedures known in the art. In some embodiments, compositions according to the first aspect can be provided as shown in the third aspect.

In a second aspect, the present invention relates to a composition for oral consumption, such as a supplement, comprising one or more vitamins, zinc, selenium, and one or more amino acids. In some embodiments, such a composition may e.g. comprise:
a. Vitamin(s); such as one or more Vitamin B, e.g. one or more of:
   Vitamin B5 (Panthoteic acid);
   Vitamin B6;
   Vitamin B7; and/or
   Vitamin B12; including any combination(s) thereof;
b. Zinc; e.g. provided as Zinc gluconate
c. Selenium; such as L-Selenomethionine; and
d. Amino acid(s); such as one or more S-comprising amino acids, e.g.:
   L-Methionine;
   L-Cysteine; and/or
   L-Cystine; including any combination(s) thereof.

Such a composition for oral consumption (also called “supplement” herein, thus both expressions can be used interchangeably) can be formulated using techniques known in the art, and may comprise accepted excipients or the like. Thus in some embodiments, a supplement can be formulated as an oral dosage form, such as pill, tablet, or capsule, and/or optionally comprising one or more excipients, anti-caking and/or flow agent.

In some embodiments, said supplement can be formulated as a capsule. In some embodiments, said capsule may optionally comprise an anti-caking and/or flow agent, such as rice bran extract. Generally, when formulated as a capsule, said capsule will comprise a capsule shell, thus in some embodiments, the supplement may comprise a capsule shell. In some embodiments, a capsule may further comprise a colouring agent. Further details e.g. concerning formulations as a capsule are given below.

A supplement as disclosed herein can e.g. be formulated such that said oral dosage form (e.g. pill, tablet, or capsule) has a weight of 0.2-2.0, 0-4-1.5, 0.6-1.0, or around 0.72 g. In some embodiments, the weight can be less than 0.2 g. In some embodiments, the weight can be more than 2.0 g.

Furthermore, a supplement according to the second aspect can be formulated for a defined number of doses per day, such as 1, 2, 3 or more. Generally, a low number of doses per day is preferred, such as 1 or 2. In some embodiments, said oral dosage form is formulated for a single dosage per day. In some embodiments, said oral dosage form is a capsule formulated for a single dosage per day.

In the following, suitable concentration ranges for the different compounds/ingredients/constituent of such a supplement, e.g. formulated as a capsule are provided.

In some embodiments, a supplement comprises one or more of 0.5-10% Vitamin(s); such as one or more Vitamin B(s); 0.1-2.0% Zinc, e.g. provided as Zinc gluconate; 0.001-2.0, Selenium; such as L-Selenomethionine; and 25-90% Amino acid(s); such as one or more S-comprising amino acids.

In some embodiments, a supplement comprises 0.5-10% Vitamin(s); such as one or more Vitamin B(s); 0.1-2.0% Zinc, e.g. provided as Zinc gluconate; 0.001-2.0, Selenium; such as L-Selenomethionine; and 25-90% Amino acid(s); such as one or more S-comprising amino acids.

In some embodiments, a supplement comprises 0.5-10% Vitamin(s); such as one or more Vitamin B(s) and/or other vitamins. In some embodiments, the concentration of vitamins can also be lower than 0.5%. In some embodiments, the concentration can also be higher than 10%.

Generally, B vitamins are believed to provide a positive effect on hair growth, skin and/or nails, as further exemplified below.

For individual B vitamins, suitable ranges may e.g. comprise by weight: 0.5-5.0, 0.8-3.0, or 1.0-1.5, or around 1.3% Vitamin B5, also known as pantothenic acid. In some embodiments, the concentration of vitamin B5 can also be lower than 0.5%. In some embodiments, the concentration of vitamin B5 can also be higher than 5%.

Vitamin B5, similar to other B vitamins, is believed to provide nourishment to the hair follicles in order for them to function properly. It strengthens and improves the hair follicle function, and a healthy hair follicle is considered important for hair growth. Vitamin B5 appears essential for hair growth and health, and a lack of vitamin B5 will lead to malnourishment of hair follicles that can result in decreased hair growth or even hair loss.

For individual B vitamins, suitable ranges may e.g. comprise by weight: 0.01-2.0, 0.1-1.5, 0.2-0.4, or around 0.28% Vitamin B6. In some embodiments, the concentration of vitamin B6 can also be lower than 0.01%. In some embodiments, the concentration of vitamin B6 can also be higher than 2%.

Vitamin B6 is believed to increase the flow of blood, such as oxygenated blood, to the scalp and/or hair follicle(s), thus supporting hair growth and/or hair revitalization. Vitamin B6 is also believed to contribute to optimum hair growth.

For individual B vitamins, suitable ranges may e.g. comprise by weight: 0.1-2.0, 0.15-1.0, 0.20-0.5, or around 0.35% Vitamin B7, also known as biotin. In some embodiments, the concentration of vitamin B7 can also be lower than 0.1%. In some embodiments, the concentration of vitamin B7 can also be higher than 2%.

Vitamin B7 is believed to stimulate keratin production in hair and/or other keratin comprising tissue(s) and can increase the rate of follicle growth.

For individual B vitamins, suitable ranges may e.g. comprise by weight: 0.00005-2.0, 0.0001-1.0, 0.0002-0.0004, or around 0.0003% Vitamin B12, also known as cobalamin. In some embodiments, the concentration of vitamin B12 can also be lower than 0.00005%. In some embodiments, the concentration of vitamin B12 can also be higher than 2%.

Vitamin B12 appears quite essential for hair growth, and lack of B12 can cause hair loss.

In some embodiments, Zinc, e.g. provided as Zinc gluconate is provided in a concentration by weight: 0.1-2.0, 0.2-1.0, 0.4-0.7, or around 0.55%. In some embodiments, the concentration of Zinc/Zinc gluconate can also be lower than 0.1%. In some embodiments, the concentration of Zinc/Zinc gluconate can also be higher than 0.55%.

Zinc is believed to play an important role in hair tissue growth and repair. It also helps keep the oil glands around the follicles working properly. Hair loss is a common symptom of zinc-deficiency.

Suitable ranges for Selenium, e.g. provided as L-Selenomethionine, may comprise 0.001-2.0, 0.005-1.0, 0.0075-0.015, or around 0.01% Selenium/Selenomethionine; In some embodiments, the concentration of Selenium/Selenomethionine can also be lower than 0.001%. In some embodiments, the concentration of vitamin B12 can also be higher than 2%.

Selenium is a trace mineral that plays many important roles in the body, including helping with hair growth.

Suitable ranges for amino acid(s) may comprise 25-90% Amino acid(s). In some embodiments, the concentration of amino acid(s) can also be lower than 25%. In some embodiments, the concentration of amino acid(s) can also be higher than 90%.

In some embodiments, said amino acids may comprise one or more essential amino acids, such as one or more selected from: phenylalanine, valine, threonine, tryptophan, methionine, leucine, isoleucine, lysine, and histidine.

Often, amino acid(s) in a supplement comprise at least one, two or three S-comprising amino acids, such as one or more of L-methionine, L-cysteine and/or L-cystine.

Suitable ranges for L-methionine may comprise 5-30, 10-25, 14-18, or around 16.7% L-Methionine. In some embodiments, the concentration of L-Methionine can also be lower than 5%. In some embodiments, the concentration of L-Methionine can also be higher than 30%.

L-methionine is believed to promote healthy skin, hair, and nails. It is also believed to be involved in the body's natural detoxification process by protecting cells from pollutants and slowing down cell aging. Moreover, it is also crucial for the absorption and bioavailability of selenium and zinc.

Suitable ranges for L-Cysteine may comprise 10-50, 15-40, 20-30, or around 25% L-Cysteine. In some embodiments, the concentration of L-Cysteine can also be lower than 10%. In some embodiments, the concentration of L-Cysteine can also be higher than 50%.

L-cysteine is believed to be the main hair growth-stimulating amino acid. A diet, poor in cysteine, and/or health problems resulting in deficiencies relating to cysteine absorption in the body, are considered a common cause of hair loss.

Suitable ranges for L-Cystine may comprise 10-50, 20-40, 26-32, or around 28% L-Cystine. In some embodiments, the concentration of L-Cystine can also be lower than 10%. In some embodiments, the concentration of L-Cystine can also be higher than 50%.

L-Cystine is a hair building block and the oxidized dimer of cysteine in keratin from hair and nail. L-cystine is the principal protein component of keratin, the main hair protein which is believed to comprise around 17.5% of L-cystine.

In some embodiments, a supplement is provided comprising at least 1, 2, 3 or 4 B vitamins, and optionally, wherein said at least 1, 2, 3, or 4 B Vitamins are provided in a concentration by weight of:
0.5-5.0, 0.8-3.0, or 1.0-1.5, or around 1.3% for Vitamin B5;
0.01-2.0, 0.1-1.5, 0.2-0.4, or around 0.28% Vitamin B6;
0.1-2.0, 0.15-1.0, 0.20-0.5, or around 0.35% for Vitamin B7; and/or
0.00005-2.0, 0.0001-1.0, 0.0002-0.0004, or around 0.0003% for Vitamin B12; and one or more of:
Zinc, Selenium, and/or amino acids.

In some embodiments, a supplement is provided comprising 0.1-2.0, 0.2-1.0, 0.4-0.7, or around 0.55% Zinc, such as Zinc gluconate; and one or more of: vitamins, selenium and/or amino acids.

In some embodiments, a supplement is provided comprising 0.001-2.0, 0.005-1.0, 0.0075-0.015, or around 0.01% Selenium; such as L-Selenomethionine; and one or more of: vitamins, zinc and/or amino acids.

In some embodiments, a supplement is provided comprising at least 1, 2 or 3 S-comprising amino acids, wherein said at least 1, 2 or 3 S-comprising amino acids are provided in a concentration by weight of:
5-30, 10-25, 14-18, or around 16.7% L-Methionine;
10-50, 15-40, 20-30, or around 25% L-Cysteine; and/or
10-50, 20-40, 26-32, or around 28% L-Cystine; and one or more of;
Vitamins, Zinc and/or Selenium.

As disclosed herein, a supplement can be formulated as a capsule. Such capsules may usually comprise 5-60% capsule-related components, such as capsule shell-related material and/or colouring agents. Furthermore, a capsule may comprise one or more further agents and/or ingredients, such as one or more flow- and/or anti-caking agent(s), such as rice bran extract. In some embodiments, a capsule may comprise one more of (by weight): 5-30% rice bran extract, 5-30% capsule shell, and/or 0.1-2% colouring agent.

Compositions for oral intake, such as those presented herein above are balanced in terms of concentration of active ingredients, the intended effect, and their applicability for an intended, daily use e.g. as a supplement, such as a supplement formulated as capsule. In some embodiments, a hair supplement can be formulated as single oral dose, e.g. as one capsule of 0.5-1.0, or around 0.72 g per day. Sometimes, due to the number and amount of further ingredients, daily doses may comprise more than on oral dosage form/capsule, such as 2, 3, or more. Examples of different suitable oral dosage forms and their weights are disclosed elsewhere.

In some embodiments, the composition is formulated as oral dosage form (e.g. capsule) wherein 1, 2 or 3 of said oral dosage forms (e.g. capsules) of 720 mg/day. In some embodiments, 1 capsule per day will provide the required daily dose. In some embodiments, 2 capsules per day will provide the required daily dose. In some embodiments, 3 capsules per day will provide the required daily dose. In some embodiments, more than 3 capsules per day will provide the require daily dose.

In some embodiments, the composition is formulated as an oral dosage form, such as capsule of around 0.5-1.0 or 0.72, wherein the daily dose for a subject, such as an adolescent, adult or senior is one or two oral dosage forms/day. In some preferred embodiments, the number of oral doses per day is one. This is generally believed as being an advantage for the subject. However, e.g. in some cases, it can be advantageous to provide the daily dose distributed over 2 or more capsules, tablets, pills or the like.

As disclosed herein, in some embodiments, supplements can be formulated for oral intake or consumption, formulated e.g. as a tablet, pill, or capsule. Such formulation(s) may comprise an acceptable excipient, such as 1-80, 2-70, 3-60, 4-50, 5-30, 8-20, 10-15, or around 12% by weight. An example of such an excipient is Rice Bran Extract. In some embodiments, said excipient may be present in a concentration of at least 5, 10, 20, 30, 40, 50, 60, 70, or 80% by weight, or more. In some embodiments, suitable ranges for rice bran extract may comprise 5-30, 6-20, 8-14, or around 11% rice bran extract. In some embodiments, the concentration of rice bran extract can also be lower than 5%. In some embodiments, the concentration of rice bran extract can also be higher than 30%.

When formulated as a capsule, said composition may comprise a capsule shell, such as conventional capsule shell. This shell may e.g. comprise 4-50, 5-30, 10-25, 15-20, or around 17% by weight capsule shell. In some embodiments, said capsule shell may comprise a glazing agent, such as Hydroxypropyl Methylcellulose, and/or a colouring agent, such as TiO₂. In some embodiments, suitable ranges for capsule shell components may be 5-30, 8-35, 15-20%, or around 17% by weight. In some embodiments, the concentration of capsule shell can also be lower than 5%. In some embodiments, the concentration of capsule shell can also be higher than 30%.

Furthermore, oral dosage forms such as capsules may comprise a colouring agent. Suitable ranges for colouring agent(s), such as TiO₂ may e.g. be 0.1-2, 0.15-1, 0.2-0.4, or around 0.3%. In some embodiments, the concentration of colouring agent(s) can also be lower than 0.1%. In some embodiments, the concentration of colouring agent(s) can also be higher than 2%.

Generally, in the embodiments presented herein, the percentages are given for compositions which may comprise an excipient and/or a capsule shell. A person skilled in the art can derive other dosages, e.g. for an oral dosage form without said excipient and/or capsule shell without undue burden.

In some embodiments, the composition is formulated as a supplement for daily intake, such as 1-5, e.g. 1, 2 or 3 pills, tablets, capsules, or the like per day. Often, daily intake will be in the morning, in combination with breakfast and/or consumption of fluid, such as 100 ml of fluid, such as water, milk, tee or coffee. Alternatively, other time points can be chosen as well, such as lunch or dinner.

In some embodiments, the composition is formulated for oral intake, such as an oral dosage form formulated as tablet, pill, or capsule with a weight of around 0.1-2.0 g, 0.4-1.5 g, 0.5-1.0 g, or around 0.72 g. In some embodiments, said daily doses forms have a weight of around 0.5, 0.6, 0.7, 0.8, 0.9, 1.0, 1.2, 1.4, 1.6, 1.8, or 2.0 g. In some embodiments, said weight can be less than 0.5 g, 0.5-0.6, 0.6-0.7, 0.7-0.8, 0.8-0.9, 0.9-1.0 g, 1.0-2.0 g, or (iv) more than 2.0 g. In some embodiments, the supplements are formulated as capsules, with a weight of around 0.6-0.8 g, such as around, 0.6, 0.625, 0.65, 0.675, 0.7, 0.71, 0.72, 0.73, 0.74, 0.75, 0.8, 0.9 or 1.0 g.

The ingredients/components/constituents of a composition can be provided in different forms, ranging from essentially pure form to more complex extract.

In some embodiments, a supplement is provided comprising (% by weight):
ci. 0.5-10% Vitamin(s); such as one or more Vitamin B(s), e.g.:
   0.5-5.0, 0.8-3.0, or 1.0-1.5, or around 1.3% Vitamin B5;
   0.01-2.0, 0.1-1.5, 0.2-0.4, or around 0.28% Vitamin B6;
   0.1-2.0, 0.15-1.0, 0.20-0.5, or around 0.35% Vitamin B7; and/or
   0.00005-2.0, 0.0001-1.0, 0.0002-0.0004, or around 0.0003% Vitamin B12; including any combination(s) thereof;
cii. 0.1-2.0, 0.2-1.0, 0.4-0.7, or around 0.55% Zinc, such as Zinc gluconate
ciii. 0.001-2.0, 0.005-1.0, 0.0075-0.015, or around 0.01% Selenium; such as L-Selenomethionine; and
civ. 25-90% Amino acid(s); such as one or more S-comprising amino acids, e.g.:
   5-30, 10-25, 14-18, or around 16.7% L-Methionine;
   10-50, 15-40, 20-30, or around 25% L-Cysteine; and/or
   10-50, 20-40, 26-32, or around 28% L-Cystine;
   including any combination(s) thereof; and optionally
cv. 5-60% capsule-related components; such as one or more of:
   5-30, 6-20, 8-14, or around 11% rice bran extract
   5-30, 8-35, 15-20%, or around 17% capsule shell, and
   optionally 0.1-2, 0.15-1, 0.2-0.4, or around 0.3% colouring agent, such as TiO₂.

In some embodiments, a supplement is provided comprising (% by weight):
ci. 0.5-10% Vitamin(s); such as one or more Vitamin B(s), e.g.:
   0.5-5.0% Vitamin B5;
   0.01-2.0% Vitamin B6;
   0.1-2.0% Vitamin B7; and/or
   0.00005-2.0 Vitamin B12; including any combination(s) thereof;
cii. 0.1-2.0% Zinc, such as Zinc gluconate
ciii. 0.001-2.0% Selenium; such as L-Selenomethionine; and
civ. 25-90% Amino acid(s); such as one or more S-comprising amino acids, e.g.:
   5-30% L-Methionine;
   10-50% L-Cysteine; and/or
   10-50% L-Cystine;
including any combination(s) thereof; and optionally
cv. 5-60% capsule-related components; such as one or more of:
   5-30% rice bran extract
   5-30% capsule shell, and optionally
   0.1-2% colouring agent, such as TiO₂.

In some embodiments, a supplement is provided comprising (% by weight):
cvi. 0.5-10% Vitamin(s); such as one or more Vitamin B(s), e.g.:
   0.5-5.0, 0.8-3.0, or 1.0-1.5, or around 1.3% Vitamin B5;
   0.01-2.0, 0.1-1.5, 0.2-0.4, or around 0.28% Vitamin B6;
   0.1-2.0, 0.15-1.0, 0.20-0.5, or around 0.35% Vitamin B7; and/or
   0.00005-2.0, 0.0001-1.0, 0.0002-0.0004, or around 0.0003% Vitamin B12; including any combination(s) thereof;
cvii. 0.1-2.0, 0.2-1.0, 0.4-0.7, or around 0.55% Zinc, such as Zinc gluconate
cviii. 0.001-2.0, 0.005-1.0, 0.0075-0.015, or around 0.01% Selenium; such as L-Selenomethionine; and
cix. 25-90% Amino acid(s); such as one or more S-comprising amino acids, e.g.:
   5-30, 10-25, 14-18, or around 16.7% L-Methionine;
   10-50, 15-40, 20-30, or around 25% L-Cysteine; and/or
   10-50, 20-40, 26-32, or around 28% L-Cystine;
including any combination(s) thereof; and optionally
cx. 5-60% capsule-related components; such as one or more of:
   5-30, 6-20, 8-14, or around 11% rice bran extract
   5-30, 8-35, 15-20%, or around 17% capsule shell, and
   optionally 0.1-2, 0.15-1, 0.2-0.4, or around 0.3% colouring agent, such as TiO₂.

In some embodiments, a supplement is provided comprising (% by weight):
cvi. :
   0.5-5.0% Vitamin B5;
   0.1-2.0% Vitamin B6;
   0.1-2.0% Vitamin B7; and
   0.00005-2.0 Vitamin B12;
cvii. 0.1-2.0% Zinc, such as Zinc gluconate
cviii. 0.001-2.0% Selenium; such as L-Selenomethionine; and
cix. :
   5-30% L-Methionine;
   10-50% L-Cysteine; and
   10-50% L-Cystine;
and optionally
cx. :
   5-30% rice bran extract
   5-30% capsule shell, and optionally
   0.1-2% colouring agent, such as TiO₂.

In a third aspect, the present invention pertains to a method for providing a composition, such as topical composition according to the first aspect, e.g. a hair serum, said method comprising the acts (or steps) of:
Providing a mixture of alcohol and water, by adding alcohol directly to water;
Providing the desired ingredients in their respective amounts;
Mixing and/or dissolving the ingredients in the alcohol/water mixture; and optionally
Adjusting pH to e.g. 5-6, such as 5.5, e.g. by the use of an organic acid and/or its salt, such as citric acid, e.g. citric acid monohydrate.

In some embodiments, mixing is provided using a rotor-stator homogenizer. Such devices comprise a fast-spinning inner rotor with a stationary outer sheath (stator) to homogenize samples through mechanical tearing, shear fluid forces, and/or cavitation.

In some embodiments, viscosity of the composition is adjusted by addition of one or more thickening agent(s), such as xanthan gum.

In some embodiments, the topical composition is formulated to a liquid to semi-liquid viscosity.

In some embodiments, the CBD is “type A CBD”. Often, the use of “type A CBD” is preferred in contrast to “type B CBD”.

In a fourth aspect, the present invention concerns a composition obtained or obtainable according to a method as provided herein, such as according to the third aspect.

In a fifth aspect, the present invention relates to a topical composition for use in the treatment of a hair-, scalp-, skin-, and/or nail-related condition in a subject, wherein said treatment comprises topical application of a composition as disclosed herein, such as a CBD-comprising composition according to the first or fourth aspect to one or more of skin, hair, scalp, nail, and/or nail bed of a subject.

In some embodiments, the topical composition is formulated for application on the scalp.

In some embodiments, the topical composition is formulated as a hair serum.

In some embodiments, the hair serum is formulated for daily use, such as once or twice per day, preferably once a day. Said use or application will require around 4 ml hair serum per application. In some embodiments, the hair serum is formulated for around 1-10, 2-6, 3-5 or around 4 ml per application. In some embodiments, the volume is 2, 3, 4, 5, 6, 7, 8, 9, 10, or more than 10 ml per application. This could e.g. be achieved by adjusting the amounts of solvents, such as alcohol/ethanol and water.

Generally, the concentration of ingredient/components for hair serums disclosed herein are based on 4 ml per application. In some embodiments, the dosage volume can be less than 4 ml per application. In some embodiments, the dosage volume can be more than 4 ml per application. Generally, use and/or application of a topical composition, such as a hair gel, comprises applying the composition to a dry and clean scalp, e.g. line by line, such as around 1 cm apart, concentrating on the areas, where thinning is most noticeable, preferably followed by some massaging to aid application, uptake and/or penetration of the active ingredients. Depending on the formulation, a volume such as around 4 ml, will be applied per application.

In some embodiments, a treatment of a hair-, scalp-, skin-, and/or nail-related condition with a topical composition, such as a hair serum may comprise intake of a supplement, such as a supplement comprising one or more of vitamin(s), amino acid(s), zinc and/or selenium. In some embodiments, the supplement comprises vitamin(s), amino acid(s), zinc and/or selenium.

Thus, in some embodiments, treatment of a hair-, scalp-, skin-, and/or nail-related condition comprises the use of a topical application as disclosed herein, such as according to the first or fourth aspect, in combination with the intake/use of a composition for oral intake as disclosed herein, such as according to the second or sixth aspect of the invention.

In a sixth aspect, the present invention pertains to a composition for oral intake, such as a supplement for use in the treatment of a hair-, skin-, scalp- and/or nail-related condition in a subject, wherein said treatment comprises intake of a supplement as disclosed herein, such as according to the second aspect.

In some embodiments, said treatment may also comprise topical application of a cannabinoid-comprising composition to said hair, skin, scalp and/or nail. In some embodiments, said cannabinoid in the topical composition is or comprises CBD in a physiological active amount.

In some embodiments, the CBD-comprising composition is a topical composition as disclosed herein, such as according to the first or fourth aspect.

In some embodiments, said CBD-comprising composition is a hair serum.

Thus, in some embodiments, treatment of a hair-, scalp-, skin-, and/or nail-related condition comprises the intake/use of a composition for oral intake as disclosed herein, such as according to the second aspect, in combination with the use of a topical application as disclosed herein, such as according to the first or fourth or fifth aspect of the invention.

In a seventh aspect, the present invention concerns a method of treatment a hair-, skin-, scalp- and/or nail-related condition, comprising topical application of a composition, such as a topical composition as disclosed herein, e.g. according to the first, fourth or fifth aspect, to one or more of skin, hair, scalp, nail, nail bed of a subject, such as a human or animal.

In some embodiments, said treatment may comprise intake of an oral dosage form, such as a supplement. In some embodiments, said oral dosage form, e.g. supplement, is a composition as disclosed herein, such as according to the second or sixth aspect.

In some embodiments, said treatment may comprise both treatment with a topical composition and intake of an oral dosage form.

In some embodiments, the subject suffers from symptom(s), condition(s), or disease(s) related to and/or manifested in alpha-keratin-comprising tissues comprising one or more of: (i) weakening nails, breaking nails, splitting nails; (ii) weakening hair, breaking hair, reduced hair stylability, splitting hair ends, reduced hair strength, reduced hair volume, hair-loss, hair-thinning (e.g. one or more of: receding front line, loss of hair on the crown of the scalp, diffuse thinning of the hair across the entire scalp), alopecia, alopecia areata, alopecia traction, telogen effluvium, anagen effluvium, baldness, male-pattern baldness, female-pattern baldness, inherited baldness and/or inherited hair thinning; and/or (iii) dandruff, seborrhoeic dermatitis, psoriasis, itchiness, burning sensation, dryness, greasiness, rash. In some embodiments, a subject suffers from one or more conditions (i). In some embodiments, a subject suffers from one or more conditions (ii). In some embodiments, a subject suffers from one or more conditions (iii). In some embodiments, a subject suffers from a combination of one or more conditions (i) and one or more conditions (ii). In some embodiments, a subject suffers from a combination of one or more conditions (i) and one or more conditions and (iii). In some embodiments, a subject suffers from a combination of one or more conditions (i) and one or more conditions (iii). In some embodiments, a subject suffers from a combination of one or more conditions (ii) and one or more conditions (iii). In some embodiments, a subject suffers from a combination of one or more conditions (i), one or more conditions (ii), and one or more conditions (iii).

In some embodiments, the subject is a male suffering from one or more of: androgenic alopecia; telogen effluvium, anagen effluvium, and/or alopecia traction.

In some embodiments, the subject is a female suffering from one or more of: androgenic alopecia; telogen effluvium, anagen effluvium, and/or alopecia traction.

In some embodiments, the topical composition is formulated as a hair serum.

In some embodiments, treatment comprise intake of the oral dosage form, such as a supplement. Such a supplement may comprise one or more of vitamin(s), amino acid(s), zinc and/or seleniume. In some embodiments, the supplement comprises vitamin(s), amino acid(s), zinc and/or selenium.

In an eight aspect, the present invention concerns a method of treatment a hair-, skin-, scalp- and/or nail-related condition, said method comprising intake of an oral dosage form, such as supplement as disclosed herein, e.g. according to the second or sixth aspect. Such treatment may further comprise application of topical composition, such as a cannabinoid-comprising, e.g. a CBD-comprising composition as disclosed herein, e.g. according to the first, fourth or fifth aspect, to said hair, skin, scalp and/or nail. In some embodiments, the CBD-comprising composition can be a hair serum.

In a ninth aspect, the present invention pertains to one or more composition(s) and/or method(s) as disclosed herein, e.g. a CBD-comprising composition for treatment according to the fifth aspect; an oral dosage form for treatment according to the sixth aspect; and/or a method according to the seventh or eight aspect, wherein said treatment is related to alpha-keratin-comprising tissue, such as one or more of: hair treatment, skin treatment, and/or nail treatment. A more detailed list of symptoms/conditions/diseases that can be treated according to the present invention are given elsewhere.

In summary, in some embodiments, a treatment relates to one or more of: hair treatment, skin treatment, and/or nail treatment.

In some embodiments, hair treatment comprises treatment against hair-loss and/or thinning hair. In some embodiments, said treatment alleviates, reduces, stops, counteracts hair thinning and/or hair loss; and/or re-establishes lost or weakened hair. In some embodiments, hair loss or hair thinning is caused by or related to one or more of: androgenic alopecia; telogen effluvium; anagen effluvium; and/or alopecia traction. In some embodiments, the subject is a male or female.

In some embodiments, treatment comprises a dosage regimen comprising applying 1-10, 2-8, 3-5, or around 4 ml of a topical composition as disclosed herein, such as according to the first, fourth, or fifth aspect, to/on/onto the scalp per application per day.

In some embodiments, said topical composition, such as a hair serum, is applied to the scalp line by line, e.g. 1 cm apart, comprising massaging to aid penetration, and optionally concentrating on the areas, where thinning is most noticeable.

In some embodiments, said topical composition, such as a hair serum, is used in a treatment comprising one or more cycles of 2-10, 3-7, 4-6, or 5 consecutive days of treatment followed by a period of 1-7, 2-4, 2, or 3 days without treatment.

In some embodiments, said topical composition, such as a hair serum, is used in a treatment comprising multiple cycles for a period of at least 2, 4, 6, 8, 10, 12, 14, 16, 20 weeks or more.

In some embodiments, in particular concerning treatments related to inherited hair thinning and/or inherited baldness, said treatment may comprises a treatment period of around 2 months, followed by 1-2 months period of non-treatment. In some embodiments, said treatment may comprise treatment cycles as disclosed above, such as 8-10 cycles of 5-day treatment followed by 1 or 2 months of non-treatment.

In case of combination treatment, i.e. topical composition+supplement, in some embodiments, intake of the supplement is not stopped during periods of non-treatment/application of the topical composition. In some embodiments, both application of the topical composition and intake of supplement are stopped during periods of non-treatment.

In summary, a treatment comprising a topical composition and/or a supplement as disclosed herein may provide one or more of:
increased hair volume;
reduction or no hair loss;
reduction of baldness;
provides a reduction or no male & female pattern baldness;
thickened and strengthened hair;
thicker and healthier hair;
improves general hair condition;
a more attractive or beautiful hair look;
more shiny hair;
softer hair;
increased vitality of one or more of hair, skin, nails;
improved hair stylability;
protection and or reduction of split hair ends;
better condition of the scalp
less or not itching scalp;
reduced or no dryness of the scalp;
reduced or no rash on the scalp;
reduced or no redness on the scalp;
reduced or no dandruff;
does not cause irritation and/or flaking of the sensitive skin on the scalp; it is believed that e.g. elevated alcohol concentrations can lead to irritation/destruction/destabilizing/of the delicate skin/scalp;
reduced or no greasy feel on the scalp;
feeling of coolness on the head;
mo or reduced feeling of burning;
improved vitality of the scalp; and or
improved condition of the scalp;
improved nail strength;
reduced or no broken nail(s);
improved appearance of nails
improved appearance of skin
improved nail and/or skin health
increased nail strength.

In a tenth aspect, the present invention relates to a receptacle comprising a composition according to any one of the preceding aspects.

In some embodiments the receptacle provides protection from visible and/or UV-light.

In some embodiments, the receptacle comprises a topical composition as disclosed herein. In some embodiment, the topical composition is formulated as a hair serum.

In some embodiments, the receptacle comprises a volume sufficient for one cycle of treatment, such as around 20 ml hair serum.

In some embodiments, the receptacle comprises dosage and/or measuring means for providing an appropriate aliquot for one treatment, such as a volume of around 4 ml aliquots.

In some embodiments, the receptacle comprising a supplement as disclosed herein.

In an eleventh aspect, the present invention pertains to a kit comprising one or more receptacle(s) according to the tenth aspect, an instruction for use, and optionally a packaging. In some embodiments the instruction for use may comprise following information: “Apply the serum to a dry and clean scalp, line by line(1 cm apart) concentrating on the areas, where thinning is most noticeable. Massage gently to aid the penetration.”

Thus, in some embodiments, the kit may comprise one or more receptacles comprising a topical composition, and one receptacle comprising a supplement.

In a twelfth aspect, the present invention concerns a CBD-comprising composition, such as a topical composition, wherein the CBD used in the formulation is crystalline. In some embodiments, said composition is a topical composition as disclosed herein, and/or in the third and/or fourth aspect. In some embodiments, the CBD is of type A (needle-like crystals) or capable of forming needle-like crystals. as disclosed in e.g. first aspect and/or in the Examples.

In an thirteenth aspect, the present invention pertains to a dosage regimen, comprising administering an oral supplement as disclosed herein in combination with a topical composition, in particular CBD-comprising topical composition as disclosed herein. In some embodiments, the CBD is of “type A”.

Hereinafter, the present invention is described in more detail and specifically with reference to the Examples, which are not intended to limit the present invention.

### EXAMPLES

In the context of the present invention, in particular in this section, “ingredient in raw material (%)” can also be understood as purity.

### Example 1—Provision of Hair Serum

CBD-comprising compositions according to the present invention can be provided using methods and/or know-how customary in the field. This can e.g. be performed as disclosed herein, such as according to the third aspect of the invention, and/or in particular, according to the following Examples.

### Example 2—Hair Serum Formulation

Hair serum formulations can be provided according to Example 1.

pH is adjusted to 5.5 using citric acid.

Percentages are % by weight.

Generally, crystalline CBD is sourced from Enecta, unless indicated otherwise

Hair serum 1 “Anti hair loss serum mini”

Formulated per ˜20 ml tube, and 4 ml per application:

| | Ingredient in | Raw | Ingredient in |
|---|---|---|---|
| | raw material | material | formulation |
| Ingredient | (%) | (g) | (%) |
| Aqua | 100 | 12.66 | 63.30 |
| Ethanol | 96 | 6.00 | 28.80 |
| Redensyl | 62.5 | 0.60 | 1.88 |
| 1,5 Pentanediol | 98 | 0.40 | 1.96 |
| Capixyl | 57 | 0.20 | 0.57 |
| CBD Crystals | 98 | 0.02 | 0.10 |
| Menthol | 99 | 0.12 | 0.59 |
| | Total | 20.0 | |

Hair serum 2 “Anti hair loss serum complete”

| | Ingredient in | Raw | Ingredient in |
|---|---|---|---|
| | raw material | material | formulation |
| Ingredient | (%) | (g) | (%) |
| Aqua | 100 | 9.04 | 45.22 |
| Ethanol | 96 | 6.00 | 28.80 |
| Propylene glycol | 98 | 1.00 | 4.90 |
| Glycerin | 98 | 1.30 | 6.37 |
| Redensyl | 62.5 | 0.60 | 1.88 |
| 1,5 Pentanediol | 98 | 0.40 | 1.96 |
| Capixyl | 57 | 0.20 | 0.57 |
| Isopropyl palmitate | 95 | 0.18 | 0.86 |
| Triethanolamine | 98 | 0.06 | 0.27 |
| CBD Crystals | 98 | 0.02 | 0.10 |
| Menthol | 99 | 0.12 | 0.59 |
| Sodium Hyaluronate | 95 | 0.20 | 0.95 |
| Arginine | 98 | 0.0004 | 0.002 |
| Panthenol | 99 | 0.54 | 2.67 |
| Caffeine | 98.5 | 0.01 | 0.05 |
| Rona Care Biotin Plus | 5 | 0.20 | 0.05 |
| Citric acid monohydrate | 99.5 | 0.08 | 0.40 |
| Xanthan Gum | 95 | 0.05 | 0.24 |
| | Total | 20.0 | |

Hair serum 3 “Placebo anti hair loss serum” (control)

| | Ingredient in | Raw | Ingredient in |
|---|---|---|---|
| | raw material | material | formulation |
| Ingredient | (%) | (g) | (%) |
| Aqua | 100 | 9.04 | 45.20 |
| Ethanol | 96 | 6.00 | 28.80 |
| Glycerin | 98 | 1.30 | 6.37 |
| Isopropyl palmitate | 95 | 0.18 | 0.86 |
| Triethanolamine | 98 | 0.06 | 0.27 |
| Menthol | 99 | 0.12 | 0.59 |
| Citric acid monohydrate | 99.5 | 0.08 | 0.40 |
| Xanthan Gum | 95 | 0.05 | 0.24 |
| | Total | 20.0 | |

### Example 3—Provision of Supplements (Mini, Complete, Placebo)

Supplements according to the present invention are provided using methods and know-how customary in the field.

All supplements are formulated as ˜720 mg capsules using Rice Bran Extract as excipient (up to 100% by weight), and a capsule shell (˜17% by weight) comprising hydroxypropyl methyl cellulose as glazing agent and TiO₂ as colouring agent). Maltodextrin is used in the placebo formulation.

### Example 4 Supplement Formulations

Supplement formulation are provided according to Example 3. Supplements 1-3 are formulated as a single dose/capsule per day, wherein 1 capsule provides the daily required dose of ingredients. Percentages are % by weight.

Supplement 1 “Anti hair loss supplement mini”

| | Ingredient in | Raw | Ingredient in |
|---|---|---|---|
| | raw material | material | formulation |
| Ingredient | (%) | (g) | (%) |
| Vitamin B6 | 100 | 2 | 0.28 |
| Vitamin B12 | 100 | 0.0025 | 0.00 |
| Biotin | 100 | 2.5 | 0.35 |
| Panthothenic acid | 100 | 9.2 | 1.28 |
| Zinc gluconate - Zinc | 100 | 4 | 0.56 |
| Capsule Shell* | 100 | 122.00 | 16.94 |
| Rice Bran Extract | 100 | 580.3 | 80.60 |
| | Total | 720.0 | |

*Capsule shell comprises Hydroxypropyl Methylcellulose (19.56 g) as glazing agent and Titanium Dioxide/E-171 (2.44 g) as colour.

Supplement 2 “Anti hair loss supplement complete”

| | Ingredient in | Raw | Ingredient in |
|---|---|---|---|
| | raw material | material | formulation |
| Ingredient | (%) | (g) | (%) |
| L-methionine | 100 | 120 | 16.67 |
| L-Cysteine | 100 | 180 | 25.00 |
| L-Cystine | 100 | 200 | 27.78 |
| Vitamin B6 | 100 | 2 | 0.28 |
| Vitamin B12 | 100 | 0.0025 | 0.0003 |
| Biotin | 100 | 2.5 | 0.35 |
| Panthothenic acid | 100 | 9.2 | 1.28 |
| Zinc gluconate | 100 | 4 | 0.56 |
| L-Selenomethionine | 100 | 0.1 | 0.01 |
| Capsule Shell* | 100 | 122.00 | 16.94 |
| Rice Bran Extract | 100 | 80.2 | 11.14 |
| | Total | 720.0 | |

*Capsule shell comprises Hydroxypropyl Methylcellulose (19.56 g) as glazing agent and Titanium Dioxide/E-171 (2.44 g) as colour.

Supplement 3 “Placebo anti hair loss supplement” (control)

| | Ingredient in | Raw | Ingredient in |
|---|---|---|---|
| | raw material | material | formulation |
| Ingredient | (%) | (g) | (%) |
| MALTODEXTRIN DE 15-20 | 100 | 510 | 70.83 |
| Capsule Shell* | 100 | 122 | 16.94 |
| Rice Bran Extract | 100 | 88 | 12.22 |
| | Total | 720.0 | |

*Capsule shell comprises Hydroxypropyl Methylcellulose (19.56 g) as glazing agent and Titanium Dioxide/E-171 (2.44 g) as colour.

### Example 5—Application of Hair Serum

Generally, hair serums are applied in the evening, aiming at using around 4 ml per application. The serum is usually applied to a dry and clean scalp, line by line approximately 1 cm apart, concentrating on the areas, where thinning is most noticeable. Some gentle massaging is used to aid distribution, penetration and/or uptake of the active ingredients.

### Example 6—Intake of Supplement

Generally, supplements are consumed in the morning, preferably in combination with breakfast, and/or intake of around 100 ml fluid or more.

### Example 7—Combination Treatment (Supplement+Hair Serum)

Combination treatments comprise intake of supplement in the morning and application of hair serum in the evening according to Examples 6 and 5, respectively.

### Example 8—Test Set-Up

Inclusion criteria for hair serum, hair supplement, and hair serum+hair supplement:
60 subjects, 50% women, 50% men, 25-55 years, all hair types.
Subjects with score 3, 3V or 4 in Hamilton-Norwood scale (concerns men)
Subjects with score I2, I3 and I4 in Ludwig scale (concerns women)

### Non-Inclusion Criteria:

Subjects who use any treatment on the studied zone.
Pregnant or breastfeeding woman or woman planning a pregnancy during the study.
Subject presenting a pathology on the studied zone (ex. severe acne, scars).
History of drug or sun hypersensitivity, recurrent dermatological diseases or recent sunburn.
Use of topical or systemic treatment during the previous weeks liable to interfere with the assessment of the tolerance of each studied product.
Subject enrolled in another study during the study period (concerning the studied zone).
Subject considered by the investigator to be likely not compliant to the protocol.
Subject used any anti-hair loss, growth hair improvement products/supplements within one month prior to the study.

Test results are presented in the following Tables A-J, Examples 9-11.

### Example 9—Results Hair Serum

TABLE A
Hair serum 1

| Test person | 1 | 2 | 3 | 4 | 5 | 6 |
|---|---|---|---|---|---|---|
| Hair thinning | Male | Female | Female | Male | Female | Female |
| according to | HNS: 3 | LS: I3 | LS: I3 | HNS: 4 | LS: I2 | LS: I3 |
| Hamilton Norwood | | | | | | |
| (HNS) & Ludwig scale | | | | | | |
| (LS) | | | | | | |
| Test period(days) | yes | Yes | yes | yes | yes | Yes |
| 141 days | | | | | | |
| Hair density before | D0: 134 | D0: 89 | D0: 109 | D0: 81 | D0: 148 | D0: 104 |
| D0 (day 0) and after | D141: 151 | D141: 101 | D141: 122 | D141:91 | D141: 162 | D141: 115 |
| D141 (Day 141) | | | | | | |
| number of hairs | | | | | | |
| Increased hair | 13 | 13 | 12 | 11 | 9 | 10 |
| density in % | | | | | | |
| Difference | 10400 | 7000 | 7600 | 5600 | 8400 | 6400 |
| (D141-D0) | | | | | | |
| average increased | | | | | | |
| amount of hair on the | | | | | | |
| scalp/600 cm₂ | | | | | | |
| Hair thickness | D0: 0.091 | D0: 0.083 | D0: 0.085 | D0: 0.070 | D0: 0.076 | D0: 0.095 |
| before D0 (day 0) and | D141: 0.096 | D141: 0.084 | D141: 0.085 | D141: 0.072 | D141: 0.081 | D141: 0.096 |
| after D141 (Day 141) | | | | | | |
| in mm | | | | | | |
| Increased hair | 6 | 1 | 0 | 3 | 7 | 2 |
| thickness in % | | | | | | |
| Medication | None | None | None | None | None | None |
| reduced during | taking | taking | taking | taking | taking | taking |
| test period? | | | | | | |

TABLE B
Hair serum 2

| Test person | 1 | 2 | 3 | 4 | 5 | 6 |
|---|---|---|---|---|---|---|
| Hair thinning | Male | Female | Male | Female | Male | Female |
| according to | HNS: 3V | LS: I3 | HNS: 3V | LS: I3 | HNS: 3V | LS: I3 |
| Hamilton Norwood | | | | | | |
| (HNS) & Ludwig scale | | | | | | |
| (LS) | | | | | | |
| Test period(days) | yes | Yes | yes | yes | yes | Yes |
| 141 days | | | | | | |
| Hair density before | D0: 99 | D0: 116 | D0: 87 | D0: 94 | D0: 89 | D0: 104 |
| D0 (day 0) and after | D141: 123 | D141: 141 | D141: 118 | D141: 118 | D141: 116 | D141: 125 |
| D141 (Day 141) | | | | | | |
| average amount of | | | | | | |
| hair/1 cm2 | | | | | | |
| Increased hair | 25 | 21 | 36 | 26 | 31 | 20 |
| density in % | | | | | | |
| Difference | 14600 | 14800 | 18800 | 14800 | 16600 | 12400 |
| (D141-D0) | | | | | | |
| average increased | | | | | | |
| amount of hair on the | | | | | | |
| scalp/600 cm₂ | | | | | | |
| Hair thickness | D0: 0.075 | D0: 0.089 | D0: 0.066 | D0: 0.062 | D0: 0.064 | D0: 0.072 |
| before D0 (day 0) and | D141: 0.084 | D141: 0.099 | D141: 0.074 | D141: 0.071 | D141: 0.071 | D141: 0.080 |
| after D141 (Day 141) | | | | | | |
| in mm | | | | | | |
| Increased hair | 12 | 12 | 12 | 14 | 12 | 11 |
| thickness in % | | | | | | |
| Medication | NA | NA | NA | NA | NA | NA |
| reduced during | | | | | | |
| test period? | | | | | | |

TABLE C
Hair serum 3 (control)

| Test person | 1 | 2 | 3 | 4 | 5 | 6 |
|---|---|---|---|---|---|---|
| Hair thinning | Male | Female | Male | Female | Female | Female |
| according to | HNS: 3V | LS: I3 | HNS: 3V | LS: I4 | LS: I3 | LS: I2 |
| Hamilton Norwood | | | | | | |
| (HNS) & Ludwig scale | | | | | | |
| (LS) | | | | | | |
| Test period(days) | yes | Yes | yes | yes | yes | Yes |
| 141 days | | | | | | |
| Hair density before | D0: 125 | D0: 113 | D0: 107 | D0: 94 | D0: 125 | D0: 160 |
| D0 (day 0) and after | D141: 132 | D141: 123 | D141: 104 | D141: 100 | D141: 133 | D141: 170 |
| D141 (Day 141) | | | | | | |
| average amount of | | | | | | |
| hair/1 cm2 | | | | | | |
| Increased hair | 6 | 8 | −3 | 6 | 6 | 6 |
| density in % | | | | | | |
| Difference | 4200 | 5700 | −2000 | 3600 | 4800 | 6200 |
| (D141-D0) | | | | | | |
| average increased | | | | | | |
| amount of hair on the | | | | | | |
| scalp/600 cm₂ | | | | | | |
| Hair thickness | D0: 0.091 | D0: 0.081 | D0: 0.066 | D0: 0.074 | D0: 0.072 | D0: 0.072 |
| before D0 (day 0) and | D141: 0.095 | D141: 0.084 | D141: 0.066 | D141: 0.077 | D141: 0.073 | D141: 0.075 |
| after D141 (Day 141) | | | | | | |
| in mm | | | | | | |
| Increased hair | 4 | 4 | 0 | 4 | 2 | 4 |
| thickness in % | | | | | | |
| Medication | NA | NA | NA | NA | NA | NA |
| reduced during | | | | | | |
| test period? | | | | | | |

### Example 10—Supplement

TABLE D
Supplement 1

| Test person | 1 | 2 | 3 | 4 | 5 | 6 |
|---|---|---|---|---|---|---|
| Hair thinning | Male | Female | Female | Male | Male | Female |
| according to | HNS: 3V | LS: I3 | LS: I3 | HNS: 3V | HNS: 3V | LS: I3 |
| Hamilton Norwood | | | | | | |
| (HNS) & Ludwig scale | | | | | | |
| (LS) | | | | | | |
| Test period(days) | yes | Yes | yes | yes | yes | yes |
| 141 days | | | | | | |
| Hair density before | D0: 126 | D0: 111 | D0: 121 | D0: 91 | D0: 92 | D0: 124 |
| D0 (day 0) and after | D141: 134 | D141: 118 | D141: 124 | D141:98 | D141: 100 | D141: 125 |
| D141 (Day 141) | | | | | | |
| number of hairs | | | | | | |
| Increased hair | 6 | 6 | 3 | 8 | 9 | 1 |
| density in % | | | | | | |
| Difference | 4800 | 4200 | 1800 | 4200 | 4800 | 600 |
| (D141-D0) | | | | | | |
| average increased | | | | | | |
| amount of hair on the | | | | | | |
| scalp/600 cm₂ | | | | | | |
| Hair thickness | D0: 0.089 | D0: 0.091 | D0: 0.078 | D0: 0.080 | D0: 0.096 | D0: 0.075 |
| before D0 (day 0) and | D141: 0.092 | D141: 0.092 | D141: 0.081 | D141: 0.083 | D141: 0.097 | D141: 0.079 |
| after D141 (Day 141) | | | | | | |
| in mm | | | | | | |
| Increased hair | 3 | 1 | 3 | 4 | 1 | 5 |
| thickness in % | | | | | | |
| Medication | None | None | None | None | None | None |
| reduced during | taking | taking | taking | taking | taking | taking |
| test period? | | | | | | |

TABLE E
Supplement 2

| Test person | 1 | 2 | 3 | 4 | 5 | 6 |
|---|---|---|---|---|---|---|
| Hair thinning | Male | Female | Female | Female | Male | Female |
| according to | HNS: 3V | LS: I3 | LS: I3 | LS: I4 | HNS: 3V | LS: I3 |
| Hamilton Norwood | | | | | | |
| (HNS) & Ludwig scale | | | | | | |
| (LS) | | | | | | |
| Test period(days) | yes | Yes | yes | yes | yes | yes |
| 141 days | | | | | | |
| Hair density before | D0: 107 | D0: 123 | D0: 96 | D0: 83 | D0: 99 | D0: 113 |
| D0 (day 0) and after | D141: 120 | D141: 137 | D141: 111 | D141: 92 | D141: 105 | D141: 121 |
| D141 (Day 141) | | | | | | |
| average amount of | | | | | | |
| hair/1 cm2 | | | | | | |
| Increased hair | 12 | 11 | 16 | 11 | 6 | 7 |
| density in % | | | | | | |
| Difference | 7800 | 8400 | 9000 | 5400 | 3600 | 4800 |
| (D141-D0) | | | | | | |
| average increased | | | | | | |
| amount of hair on the | | | | | | |
| scalp/600 cm₂ | | | | | | |
| Hair thickness | D0: 0.063 | D0: 0.077 | D0: 0.087 | D0: 0.065 | D0: 0.071 | D0: 0.081 |
| before D0 (day 0) and | D141: 0.069 | D141: 0.084 | D141: 0.095 | D141: 0.069 | D141: 0.074 | D141: 0.086 |
| after D141 (Day 141) | | | | | | |
| in mm | | | | | | |
| Increased hair | 10 | 9 | 9 | 6 | 4 | 6 |
| thickness in % | | | | | | |
| Medication | NA | NA | NA | NA | NA | NA |
| reduced during | | | | | | |
| test period? | | | | | | |

TABLE F
Supplement 3 (control)

| Test person | 1 | 2 | 3 | 4 | 5 | 6 |
|---|---|---|---|---|---|---|
| Hair thinning | Female | Female | Male | Female | Male | Male |
| according to | LS: I3 | LS: I3 | HNS: 3V | LS: I3 | HNS: 3V | HNS: 3 |
| Hamilton Norwood | | | | | | |
| (HNS) & Ludwig scale | | | | | | |
| (LS) | | | | | | |
| Test period(days) | yes | Yes | yes | yes | yes | yes |
| 141 days | | | | | | |
| Hair density before | D0: 125 | D0: 107 | D0: 121 | D0: 98 | D0: 116 | D0: 141 |
| D0 (day 0) and after | D141: 127 | D141: 111 | D141: 121 | D141: 100 | D141: 117 | D141: 144 |
| D141 (Day 141) | | | | | | |
| average amount of | | | | | | |
| hair/1 cm2 | | | | | | |
| Increased hair | 2 | 4 | 0 | 2 | 1 | 2 |
| density in % | | | | | | |
| Difference | 1200 | 2400 | 0 | 1200 | 600 | 1800 |
| (D141-D0) | | | | | | |
| average increased | | | | | | |
| amount of hair on the | | | | | | |
| scalp/600 cm₂ | | | | | | |
| Hair thickness | D0: 0.072 | D0: 0.086 | D0: 0.071 | D0: 0.086 | D0: 0.065 | D0: 0.077 |
| before D0 (day 0) and | D141: 0.072 | D141: 0.087 | D141: 0.072 | D141: 0.087 | D141: 0.065 | D141: 0.076 |
| after D141 (Day 141) | | | | | | |
| In mm | | | | | | |
| Increased hair | 0 | 1 | 1 | 1 | 0 | −1 |
| thickness in % | | | | | | |
| Medication | NA | NA | NA | NA | NA | NA |
| reduced during | | | | | | |
| test period? | | | | | | |

### Example 11—Combination Treatment

TABLE G
Hair serum 2 + supplement 1

| Test person | 1 | 2 | 3 | 4 | 5 | 6 |
|---|---|---|---|---|---|---|
| Hair thinning | Female | Female | Male | Male | Female | Male |
| according to | LS: I3 | LS: I4 | HNS: 3V | HNS: 3 | LS: I4 | HNS: 3 |
| Hamilton Norwood | | | | | | |
| (HNS) & Ludwig scale | | | | | | |
| (LS) | | | | | | |
| Test period(days) | yes | Yes | yes | yes | yes | yes |
| 141 days | | | | | | |
| Hair density before | D0: 110 | D0: 79 | D0: 99 | D0: 124 | D0: 89 | D0: 141 |
| D0 (day 0) and after | D141: 136 | D141:96 | D141: 125 | D141: 153 | D141: 121 | D141: 168 |
| D141 (Day 141) | | | | | | |
| number of hairs | | | | | | |
| Increased hair | 24 | 22 | 26 | 23 | 36 | 19 |
| density in % | | | | | | |
| Difference | 15600 | 16200 | 15600 | 17400 | 19200 | 16200 |
| (D141-D0) | | | | | | |
| average increased | | | | | | |
| amount of hair on the | | | | | | |
| scalp/600 cm₂ | | | | | | |
| Hair thickness | D0: 0.083 | D0: 0.077 | D0: 0.066 | D0: 0.081 | D0: 0.086 | D0: 0.075 |
| before D0 (day 0) and | D141: 0.091 | D141: 0.086 | D141: 0.074 | D141: 0.092 | D141: 0.094 | D141: 0.086 |
| after D141 (Day 141) | | | | | | |
| in mm | | | | | | |
| Increased hair | 10 | 12 | 12 | 14 | 9 | 15 |
| thickness in % | | | | | | |
| Medication | NA | NA | NA | NA | NA | NA |
| reduced during | | | | | | |
| test period? | | | | | | |

TABLE H
Hair serum 2 + supplement 2

| Test person | 1 | 2 | 3 | 4 | 5 | 6 |
|---|---|---|---|---|---|---|
| Hair thinning | Female | Female | Male | Female | Male | Female |
| according to | LS: I3 | LS: I3 | HNS: 3V | LS: I4 | HNS: 3V | LS: I4 |
| Hamilton Norwood | | | | | | |
| (HNS) & Ludwig scale | | | | | | |
| (LS) | | | | | | |
| Test period(days) | yes | Yes | yes | yes | yes | yes |
| 141 days | | | | | | |
| Hair density before | D0: 97 | D0: 105 | D0: 99 | D0: 78 | D0: 103 | D0: 86 |
| D0 (day 0) and after | D141: 131 | D141: 143 | D141: 139 | D141: 114 | D141: 146 | D141: 125 |
| D141 (Day 141) | | | | | | |
| average amount of | | | | | | |
| hair/1 cm2 | | | | | | |
| Increased hair | 35 | 36 | 40 | 46 | 42 | 45 |
| density in % | | | | | | |
| Difference | 20400 | 22800 | 24000 | 21600 | 25800 | 23400 |
| (D141-D0) | | | | | | |
| average increased | | | | | | |
| amount of hair on the | | | | | | |
| scalp/600 cm₂ | | | | | | |
| Hair thickness | D0: 0.073 | D0: 0.066 | D0: 0.081 | D0: 0.092 | D0: 0.084 | D0: 0.070 |
| before D0 (day 0) and | D141: 0.085 | D141: 0.079 | D141: 0.093 | D141: 0.098 | D141: 0.095 | D141: 0.078 |
| after D141 (Day 141) | | | | | | |
| in mm | | | | | | |
| Increased hair | 16 | 20 | 15 | 7 | 13 | 11 |
| thickness in % | | | | | | |
| Medication | NA | NA | NA | NA | NA | NA |
| reduced during | | | | | | |
| test period? | | | | | | |

TABLE J
Hair serum 2 + supplement 3 (control)

| Test person | 1 | 2 | 3 | 4 | 5 | 6 |
|---|---|---|---|---|---|---|
| Hair thinning | Male | Male | Male | Female | Female | Female |
| according to | HNS: 3V | HNS: 3V | HNS: 3V | LS: I3 | LS: I2 | LS: I2 |
| Hamilton Norwood | | | | | | |
| (HNS) & Ludwig scale | | | | | | |
| (LS) | | | | | | |
| Test period(days) | yes | yes | yes | yes | yes | yes |
| 141 days | | | | | | |
| Hair density before | D0: 101 | D0: 97 | D0: 124 | D0: 91 | D0: 117 | D0: 146 |
| D0 (day 0) and after | D141: 131 | D141: 125 | D141: 149 | D141: 118 | D141: 146 | D141: 171 |
| D141 (Day 141) | | | | | | |
| average amount of | | | | | | |
| hair/1 cm2 | | | | | | |
| Increased hair | 30 | 29 | 20 | 30 | 25 | 17 |
| density in % | | | | | | |
| Difference | 18000 | 16800 | 15000 | 16200 | 17400 | 15000 |
| (D141-D0) | | | | | | |
| average Increased | | | | | | |
| amount of hair on the | | | | | | |
| scalp/600 cm₂ | | | | | | |
| Hair thickness | D0: 0.082 | D0: 0.081 | D0: 0.069 | D0: 0.089 | D0: 0.069 | D0: 0.091 |
| before D0 (day 0) and | D141: 0.091 | D141: 0.087 | D141: 0.078 | D141: 0.093 | D141: 0.078 | D141: 0.098 |
| after D141 (Day 141) | | | | | | |
| in mm | | | | | | |
| Increased hair | 11 | 7 | 13 | 5 | 13 | 8 |
| thickness in % | | | | | | |
| Medication | NA | NA | NA | NA | NA | NA |
| reduced during | | | | | | |
| test period? | | | | | | |

### Example 12—Provision of CBD by Alcohol Extraction, Distillation and Crystallization

Crystalline CBD can be provided by methods and techniques known in the art, such as by methods disclosed in U.S. Ser. No. 10/413,845 and/or U.S. Ser. No. 10/414,709.

In short, crystalline CBD can be provided from hemp or cannabis (Cannabis sativa) by a method consisting essentially of:
Extracting hemp or cannabis with a solvent selected from the group consisting of propanol, isopropanol, butanol, pentanol, hexanol, heptanol, and octanol to produce an extract consisting essentially of an extracted hemp or cannabis consisting essentially of tetrahydrocannabinol, a terpene, or cannabidiol;
Evaporating the solvent portion of the extract to generate a substantially solvent-free extract comprising CBD;
Distilling the substantially solvent-free extract to isolate the CBD, and
Crystallizing the distilled, isolated CBD to produce a crystallized, isolated CBD.

Often, the crystallized, isolated CBD is subjected to vacuum drying to remove volatile remnants, in particular the solvent used in crystallizing or re-crystallizing, if needed.

In particular, a method comprising extraction with isopropanol and crystallization by the use of heptane, including one or more optional re-crystallization steps, followed by vacuum drying can provide CBD with crystal structure A, i.e. needle like crystals. Furthermore, such a CBD can be very low in undesired compounds, such as terpenes.

GC chromatography or other analytical methods known in the art can be used to monitor the process such as to ensure a high yield and/or a high purity of the desired product.

Concerning the raw material, hemp comprising e.g. 2-3% CBD is dried and ground before extraction with isopropanol, such as food grade isopropanol.

Guidance for choosing the appropriate reaction based on the boiling points or ranges of the different compounds can e.g. be found here:
www.nwsci.com/customer/docs/SKUDocs/RMR/Technical %20Data Extractions 03.28.18.pdf.

CBD with crystal structure A can e.g. be provided from www.enecta.com, and/or following a similar extraction and/or purification protocol as said manufacturer.

### Example 13—Comparison of Hair Serum Compositions Formulated with Different Crystalline CBDs

Two hair serum compositions are prepared according to Example 2, the only difference being that the crystalline CBD used in the formulation is either of type A (needle-like crystals; FIG. 1) or type B (bunch/cluster-like; FIG. 2).

Type A crystalline CBD is sourced from Enecta, while type B CBD is sourced from Pharma Hemp.

When testing both hair serum compositions, surprisingly and unexpectedly, it is seen and/or it can be concluded that type A CBD is significantly more active than type B CBD.

1.-62. (canceled)

63. A composition comprising:
Cannabidiol (CBD);
Redensyl®;
Capixyl®; and
a C3 to C8 diol.

64. The composition according to claim 63, wherein:
Redensyl® comprises dihydroquercetin-glucoside, epigallotechin gallate-glucoside, glycine, zinc chloride, meta-bisulfite, and glycerin; and
Capixyl® comprises butylene glycol, dextran, acetyl tetrapeptide-3, Trifolium pratense flower extract.

65. The composition according to claim 63, wherein the C3 to C8 diol comprises 1, 5 pentanediol.

66. The composition according to claim 63 comprising:
menthol;
a skin healing compound comprising hyaluronic acid/hyaluronate;
caffeine;
propylene glycol;
a skin conditioning agent or emollient, a binder comprising isopropyl palmitate, or both a binder and a skin conditioning agent or emollient;
triethanolamine;
arginine;
panthenol;
EtOH;
a vitamin comprising a B vitamin, wherein the B vitamin comprises vitamin B7; an adjusting agent comprising citric acid monohydrate;
a skin healing compound comprising hyaluronic acid/hyaluronate;
a thickening agent comprising Xanthan gum; or
any combination thereof.

67. The composition according to claim 65 comprising a concentration (% by weight) of:
0.001-2.0, 0.05-0.5, 0.075-0.2, or around 0.1% CBD;
10-60, 30-55, 40-50, or around 30% EtOH;
0.5-5.0, 1.0-4.0, 1.5-2.5, or around 2% C3-C8 diol;
0.1-2.0, 0.2-1.0, 0.4-0.8, or around 0.6% menthol;
0.01-2.0, 0.1-1.5, 0.85-1.15, or around 0.95% of a skin healing compound comprising hyaluronic acid/hyaluronate;
0.01-2.0%, 0.02-0.5, 0.04-0.07, or around 0.05% caffeine;
1.0-20, 2-10, 4-6, or around 5.0% propylene glycol;
1.0-20, 2-15, 5-8, or around 6.5% glycerin;
0.05-2.0, 0.1-1.0, 0.7-1.2, or 0.9% of a moisturizer, an emollient, a binder comprising isopropyl palmitate, or any combination of a moisturizer, an emollient and a binder;
0.1-2.0, 0.15-1.0, 0.2-0.4, or around 0.28% triethanolamine;
0.0001-1.0, 0.0005-0.1, 0.001-0.003, or around 0.002% arginine;
0.5-5.0, 1.0-4.0, 2.5-3.0, or around 2.7% panthenol;
0.1-2.0, 0.2-1.5, 0.8-1.2, or around 1.0% of a vitamin B;
0.1-2.0, 0.15-1.0, 0.15-0.4, or around 0.25% of a thickening agent;
or any combination thereof.

68. The composition of claim 63 comprising an amount of water suitable for formulating a hair serum with the composition, wherein the amount of water suitable for formulating a hair serum with the composition is 30-70%, 35-60%, 40-55%, or around 45% water.

69. The composition of claim 63 essentially free of cannabinoids other than Cannabidiol (CBD).

70. The composition of claim 63 wherein said composition is essentially free of one or more cannabinoids comprising:
THC (tetrahydrocannabinol), THCA (tetrahydrocannabinolic acid), CBDA (cannabidiolic acid), CBN (cannabinol), CBG (cannabigerol), CBC (cannabichromene), CBL (cannabicyclol), CBV (cannabivarin), THCC (tetrahydrocannabiorcol), THCV (tetrahydrocannabivarin), THCP (tetrahydrocannabiphorol), CBDV (cannabidivarin), CBCV (cannabichromevarin), CBGV (cannabigerovarin), CBGM (cannabigerol monomethyl ether), CBE (cannabielsoin), CBT (cannabicitran), including one or more cannabinoids of the following types: CBG-type, CBC-type, CBD-type other than CBD, THC-type, CBN-type, CBE-type, iso-THC-type, CBL-type, CBT-type, or any combination thereof.

71. The composition of claim 63 comprising:
one or more cannabinoids in addition to CBD provided in an amount below 10, 5, 2, 1, 0.5, or 0.1% by weight in relation to % by weight of CBD in the composition; or
one or more cannabinoids in addition to CBD provided in a ratio to CBD in the composition of: >100:1, 100:1-50:1, 50:1-25:1, 25:1-10:1, 10:1, 10:1-5:1, 5:1-2:1, 2:1-1:1, 1:1-1:2, 1:2-1:5, or <1:5.

72. A method for treatment of an affected surface on a subject having a hair, scalp, skin, or nail condition comprising:
providing a composition as defined in claim 63 formulated as a topical preparation to the affected surface on the subject, wherein said subject is a human or non-human animal; and
providing a treatment to the affected surface of the subject.

73. The method of claim 72, wherein the topical preparation is formulated as a hair serum, and the hair, scalp, skin, or nail condition of the subject is:
Androgenic Alopecia;
Telogen effluvium;
Anagen effluvium; or
Alopecia traction.

74. The method of claim 72 further comprising providing a supplement to the subject, wherein said supplement comprises a vitamin, an amino acid, zinc, selenium, or any combination thereof, and is formulated as an oral dosage form.

75. The method of claim 74 wherein the supplement comprises:
B vitamins comprising Vitamin B5 (Pantothenic acid), Vitamin B6, Vitamin B7, Vitamin B12, or any combination thereof;
Zinc comprising Zinc gluconate;
Selenium comprising L-Selenium methionine; and
Amino acids comprising an S-amino acid, wherein said S-amino acid comprises L-Methionine, L-Cysteine, L-Cystine, or any combination thereof.

76. The method of claim 75, wherein said supplement comprises one or more of the following ingredients in a % by weight of:
0.5-10% Vitamin B, said Vitamin B comprising:
0.5-5.0, 0.8-3.0, or 1.0-1.5, or around 1.3% Vitamin B5;
0.01-2.0, 0.1-1.5, 0.2-0.4, or around 0.28% Vitamin B6;
0.1-2.0, 0.15-1.0, 0.20-0.5, or around 0.35% Vitamin B7;
0.00005-2.0, 0.0001-1.0, 0.0002-0.0004, or around 0.0003% Vitamin B12; or any combination thereof;
0.1-2.0, 0.2-1.0, 0.4-0.7, or around 0.55% Zinc comprising Zinc gluconate;
0.001-2.0, 0.005-1.0, 0.0075-0.015, or around 0.01% Selenium comprising L-Selenium methionine; and
25-90% Amino acid(s) comprising one or more S-amino acids, said S-amino acids comprising:
5-30, 10-25, 14-18, or around 16.7% L-Methionine;
10-50, 15-40, 20-30, or around 25% L-Cysteine;
10-50, 20-40, 26-32, or around 28% L-Cystine; or
any combination thereof.

77. A method of enhancing hair density on a subject scalp of a subject having a condition associated with a reduced hair density compared to hair density in a subject without the condition, comprising:
preparing a hair serum formulation comprising the composition of claim 63;
administering a treatment regimen to the subject comprising applying a pharmacologically effective amount of the hair serum formulation to the subject scalp for an effective period of time; and
enhancing hair density on the subject scalp,
wherein hair density on the subject scalp of the subject administered the treatment regimen is enhanced compared to hair density on a subject scalp having a condition associated with reduced hair density without the treatment regimen.

78. The method of claim 77 wherein the treatment regimen comprises:
one or more treatment cycles of administering an effective amount of around 1-10 ml, 2-8 ml, 3-5 ml, or around 4 ml of said hair serum formulation to the subject scalp, applied on the subject scalp at about 1 cm spaced intervals; and
massaging the scalp.

79. The method of claim 77 wherein the treatment regimen comprises one or more treatment cycles of 2-10, 3-7, 4-6, or 5 consecutive days of treatment with said hair serum formulation to the subject scalp, followed by a period of 1-7, 2-4, 2 or 3 days without treatment with said hair serum formulation to the subject scalp.

80. The method of claim 77 wherein the treatment regimen comprises:
around 60 consecutive days of application of the hair serum formulation to the subject scalp, followed by 30 to 60 days without application of the hair serum formulation to the subject scalp; or
8-10 treatment cycles of a 5-day application of the hair serum formulation to the subject scalp followed by 2 days without application of the hair serum formulation where the subject condition is an inherited hair thinning or inherited baldness condition.

81. A method for preparing a composition suitable for use in treatment of a skin condition, comprising:
combining an amount of water and an amount of alcohol to provide a mixture; and
adding to the mixture one or more ingredients to provide the composition, wherein said ingredients comprise:
Cannabidiol (CBD);
Redensyl®;
Capixyl®; and
a C3 to C8 diol.

82. The method of claim 81 wherein the composition comprises an amount of water suitable to formulate a hair serum comprising the composition.

83. The method of claim 81 wherein the one or more ingredients added to the mixture comprise:
0.001-2.0, 0.05-0.5, 0.075-0.2, or around 0.1% CBD;
10-60, 30-55, 40-50, or around 30% EtOH;
0.5-5.0, 1.0-4.0, 1.5-2.5, or around 2% of a C3-C8 diol;
0.1-2.0, 0.2-1.0, 0.4-0.8, or around 0.6% menthol;
0.01-2.0, 0.1-1.5, 0.85-1.15, or around 0.9% of a skin healing compound comprising hyaluronic acid/hyaluronate;
0.01-2.0%,0.02-0.5, 0.04-0.07, or around 0.05% caffeine;
1.0-20, 2-10, 4-6, or around 5.0% propylene glycol;
1.0-20, 2-15, 5-8, or around 6.5% glycerin;
0.05-2.0, 0.1-1.0, 0.7-1.2, or 0.9% of a moisturizer, an emollient, a binder comprising Isopropyl palmitate, or any combination of a moisturizer, an emollient and a binder;
0.1-2.0, 0.15-1.0, 0.2-0.4, or around 0.28% triethanolamine;
0.0001-1.0, 0.0005-0.1, 0.001-0.003, or around 0.002% arginine;
0.5-5.0, 1.0-4.0, 2.5-3.0, or around 2.7% panthenol;
0.1-2.0, 0.2-1.5, 0.8-1.2, or around 1.0% of a vitamin B;
0.1-2.0, 0.15-1.0, 0.15-0.4, or around 0.25% of a thickening agent; or any combination thereof.
